SEQUENCE LISTING (263 sequences)

SEQ ID NO: 1 - DNA, 20 bp, synthetic construct
  misc_feature 1..20: note = None
  source 1..20: mol_type = other DNA
gggctataagaggagccggg

SEQ ID NO: 2 - DNA, 20 bp, synthetic construct
  misc_feature 1..20: note = None
  source 1..20: mol_type = other DNA
gaggccgttgcattgcccca

SEQ ID NO: 3 - DNA, 20 bp, synthetic construct
  misc_feature 1..20: note = None
  source 1..20: mol_type = other DNA
ctcactttcacccaggctca

SEQ ID NO: 4 - DNA, 20 bp, synthetic construct
  misc_feature 1..20: note = None
  source 1..20: mol_type = other DNA
cggggaactcactttcaccc

SEQ ID NO: 5 - DNA, 20 bp, synthetic construct
  misc_feature 1..20: note = None
  source 1..20: mol_type = other DNA
ggtgaaagtgagttccccgt

SEQ ID NO: 6 - DNA, 20 bp, synthetic construct
  misc_feature 1..20: note = None
  source 1..20: mol_type = other DNA
gaaagtgagttccccgttgg

SEQ ID NO: 7 - DNA, 20 bp, synthetic construct
  misc_feature 1..20: note = None
  source 1..20: mol_type = other DNA
ctcgtctgttgcctccaacg

SEQ ID NO: 8 - DNA, 20 bp, synthetic construct
  misc_feature 1..20: note = None
  source 1..20: mol_type = other DNA
cctcgtctgttgcctccaac

SEQ ID NO: 9 - DNA, 20 bp, synthetic construct
  misc_feature 1..20: note = None
  source 1..20: mol_type = other DNA
tcctcgtctgttgcctccaa

SEQ ID NO: 10 - DNA, 20 bp, synthetic construct
  misc_feature 1..20: note = None
  source 1..20: mol_type = other DNA
cccgttggaggcaacagacg

SEQ ID NO: 11 - DNA, 20 bp, synthetic construct
  misc_feature 1..20: note = None
  source 1..20: mol_type = other DNA
tggaggcaacagacgaggag

SEQ ID NO: 12 - DNA, 20 bp, synthetic construct
  misc_feature 1..20: note = None
  source 1..20: mol_type = other DNA
ggcaacagacgaggagagga

SEQ ID NO: 13 - DNA, 20 bp, synthetic construct
  misc_feature 1..20: note = None
  source 1..20: mol_type = other DNA
acagacgaggagaggatgga

SEQ ID NO: 14 - DNA, 20 bp, synthetic construct
  misc_feature 1..20: note = None
  source 1..20: mol_type = other DNA
cgaggagaggatggaaggcc

SEQ ID NO: 15 - DNA, 20 bp, synthetic construct
  misc_feature 1..20: note = None
  source 1..20: mol_type = other DNA
agggctcattcttgggggcc

SEQ ID NO: 16 - DNA, 20 bp, synthetic construct
  misc_feature 1..20: note = None
  source 1..20: mol_type = other DNA
acctcagggctcattcttgg

SEQ ID NO: 17 - DNA, 20 bp, synthetic construct
  misc_feature 1..20: note = None
  source 1..20: mol_type = other DNA
aacctcagggctcattcttg

SEQ ID NO: 18 - DNA, 20 bp, synthetic construct
  misc_feature 1..20: note = None
  source 1..20: mol_type = other DNA
gaacctcagggctcattctt

SEQ ID NO: 19 - DNA, 20 bp, synthetic construct
  misc_feature 1..20: note = None
  source 1..20: mol_type = other DNA
tgaacctcagggctcattct

SEQ ID NO: 20 - DNA, 20 bp, synthetic construct
  misc_feature 1..20: note = None
  source 1..20: mol_type = other DNA
gcccccaagaatgagccctg

SEQ ID NO: 21 - DNA, 20 bp, synthetic construct
  misc_feature 1..20: note = None
  source 1..20: mol_type = other DNA
cagccgctccctgaacctca

SEQ ID NO: 22 - DNA, 20 bp, synthetic construct
  misc_feature 1..20: note = None
  source 1..20: mol_type = other DNA
ccagccgctccctgaacctc

SEQ ID NO: 23 - DNA, 20 bp, synthetic construct
  misc_feature 1..20: note = None
  source 1..20: mol_type = other DNA
aagaatgagccctgaggttc

SEQ ID NO: 24 - DNA, 20 bp, synthetic construct
  misc_feature 1..20: note = None
  source 1..20: mol_type = other DNA
agaatgagccctgaggttca

SEQ ID NO: 25 - DNA, 20 bp, synthetic construct
  misc_feature 1..20: note = None
  source 1..20: mol_type = other DNA
gagccctgaggttcagggag

SEQ ID NO: 26 - DNA, 20 bp, synthetic construct
  misc_feature 1..20: note = None
  source 1..20: mol_type = other DNA
cctgaggttcagggagcggc

SEQ ID NO: 27 - DNA, 20 bp, synthetic construct
  misc_feature 1..20: note = None
  source 1..20: mol_type = other DNA
agggagcggctggagtgagc

SEQ ID NO: 28 - DNA, 20 bp, synthetic construct
  misc_feature 1..20: note = None
  source 1..20: mol_type = other DNA
gctggacggagatctggggc

SEQ ID NO: 29 - DNA, 20 bp, synthetic construct
  misc_feature 1..20: note = None
  source 1..20: mol_type = other DNA
cgcagctggacggagatctg

SEQ ID NO: 30 - DNA, 20 bp, synthetic construct
  misc_feature 1..20: note = None
  source 1..20: mol_type = other DNA
ccgcagctggacggagatct

SEQ ID NO: 31 - DNA, 20 bp, synthetic construct
  misc_feature 1..20: note = None
  source 1..20: mol_type = other DNA
cccgcagctggacggagatc

SEQ ID NO: 32 - DNA, 20 bp, synthetic construct
  misc_feature 1..20: note = None
  source 1..20: mol_type = other DNA
ctctgggacccgcagctgga

SEQ ID NO: 33 - DNA, 20 bp, synthetic construct
  misc_feature 1..20: note = None
  source 1..20: mol_type = other DNA
cccagatctccgtccagctg

SEQ ID NO: 34 - DNA, 20 bp, synthetic construct
  misc_feature 1..20: note = None
  source 1..20: mol_type = other DNA
ccagatctccgtccagctgc

SEQ ID NO: 35 - DNA, 20 bp, synthetic construct
  misc_feature 1..20: note = None
  source 1..20: mol_type = other DNA
aggcctctgggacccgcagc

SEQ ID NO: 36 - DNA, 20 bp, synthetic construct
  misc_feature 1..20: note = None
  source 1..20: mol_type = other DNA
cgtccagctgcgggtcccag

SEQ ID NO: 37 - DNA, 20 bp, synthetic construct
  misc_feature 1..20: note = None
  source 1..20: mol_type = other DNA
cgagtgtaacccaggcctct

SEQ ID NO: 38 - DNA, 20 bp, synthetic construct
  misc_feature 1..20: note = None
  source 1..20: mol_type = other DNA
agctgcgggtcccagaggcc

SEQ ID NO: 39 - DNA, 20 bp, synthetic construct
  misc_feature 1..20: note = None
  source 1..20: mol_type = other DNA
gcgagtgtaacccaggcctc

SEQ ID NO: 40 - DNA, 20 bp, synthetic construct
  misc_feature 1..20: note = None
  source 1..20: mol_type = other DNA
gctgcgggtcccagaggcct

SEQ ID NO: 41 - DNA, 20 bp, synthetic construct
  misc_feature 1..20: note = None
  source 1..20: mol_type = other DNA
aggagctgcgagtgtaaccc

SEQ ID NO: 42 - DNA, 20 bp, synthetic construct
  misc_feature 1..20: note = None
  source 1..20: mol_type = other DNA
tgggttacactcgcagctcc

SEQ ID NO: 43 - DNA, 20 bp, synthetic construct
  misc_feature 1..20: note = None
  source 1..20: mol_type = other DNA
gggttacactcgcagctcct

SEQ ID NO: 44 - DNA, 20 bp, synthetic construct
  misc_feature 1..20: note = None
  source 1..20: mol_type = other DNA
ggttacactcgcagctcctg

SEQ ID NO: 45 - DNA, 20 bp, synthetic construct
  misc_feature 1..20: note = None
  source 1..20: mol_type = other DNA
gttacactcgcagctcctgg

SEQ ID NO: 46 - DNA, 20 bp, synthetic construct
  misc_feature 1..20: note = None
  source 1..20: mol_type = other DNA
acactcgcagctcctggggg

SEQ ID NO: 47 - DNA, 20 bp, synthetic construct
  misc_feature 1..20: note = None
  source 1..20: mol_type = other DNA
gcacgtcaagggcctccccc

SEQ ID NO: 48 - DNA, 20 bp, synthetic construct
  misc_feature 1..20: note = None
  source 1..20: mol_type = other DNA
tgggaactgaggcacgtcaa

SEQ ID NO: 49 - DNA, 20 bp, synthetic construct
  misc_feature 1..20: note = None
  source 1..20: mol_type = other DNA
ttgggaactgaggcacgtca

SEQ ID NO: 50 - DNA, 20 bp, synthetic construct
  misc_feature 1..20: note = None
  source 1..20: mol_type = other DNA
gggttcctgtttgggaactg

SEQ ID NO: 51 - DNA, 20 bp, synthetic construct
  misc_feature 1..20: note = None
  source 1..20: mol_type = other DNA
acgtgcctcagttcccaaac

SEQ ID NO: 52 - DNA, 20 bp, synthetic construct
  misc_feature 1..20: note = None
  source 1..20: mol_type = other DNA
ccttcccagggttcctgttt

SEQ ID NO: 53 - DNA, 20 bp, synthetic construct
  misc_feature 1..20: note = None
  source 1..20: mol_type = other DNA
tccttcccagggttcctgtt

SEQ ID NO: 54 - DNA, 20 bp, synthetic construct
  misc_feature 1..20: note = None
  source 1..20: mol_type = other DNA
cagttcccaaacaggaaccc

SEQ ID NO: 55 - DNA, 20 bp, synthetic construct
  misc_feature 1..20: note = None
  source 1..20: mol_type = other DNA
agttcccaaacaggaaccct

SEQ ID NO: 56 - DNA, 20 bp, synthetic construct
  misc_feature 1..20: note = None
  source 1..20: mol_type = other DNA
cccaaacaggaaccctggga

SEQ ID NO: 57 - DNA, 20 bp, synthetic construct
  misc_feature 1..20: note = None
  source 1..20: mol_type = other DNA
cacttctctggtccttccca

SEQ ID NO: 58 - DNA, 20 bp, synthetic construct
  misc_feature 1..20: note = None
  source 1..20: mol_type = other DNA
gcacttctctggtccttccc

SEQ ID NO: 59 - DNA, 20 bp, synthetic construct
  misc_feature 1..20: note = None
  source 1..20: mol_type = other DNA
ctgcgcaataggcacttctc

SEQ ID NO: 60 - DNA, 20 bp, synthetic construct
  misc_feature 1..20: note = None
  source 1..20: mol_type = other DNA
tcgggcactcactgcgcaat

SEQ ID NO: 61 - DNA, 20 bp, synthetic construct
  misc_feature 1..20: note = None
  source 1..20: mol_type = other DNA
cggccacatgcagctgtgtc

SEQ ID NO: 62 - DNA, 20 bp, synthetic construct
  misc_feature 1..20: note = None
  source 1..20: mol_type = other DNA
ccggccacatgcagctgtgt

SEQ ID NO: 63 - DNA, 20 bp, synthetic construct
  misc_feature 1..20: note = None
  source 1..20: mol_type = other DNA
gtgcccgacacagctgcatg

SEQ ID NO: 64 - DNA, 20 bp, synthetic construct
  misc_feature 1..20: note = None
  source 1..20: mol_type = other DNA
ccgacacagctgcatgtggc

SEQ ID NO: 65 - DNA, 20 bp, synthetic construct
  misc_feature 1..20: note = None
  source 1..20: mol_type = other DNA
tacccagggccctgtgatac

SEQ ID NO: 66 - DNA, 20 bp, synthetic construct
  misc_feature 1..20: note = None
  source 1..20: mol_type = other DNA
ctgcatgtggccggtatcac

SEQ ID NO: 67 - DNA, 20 bp, synthetic construct
  misc_feature 1..20: note = None
  source 1..20: mol_type = other DNA
tgcatgtggccggtatcaca

SEQ ID NO: 68 - DNA, 20 bp, synthetic construct
  misc_feature 1..20: note = None
  source 1..20: mol_type = other DNA
tggccggtatcacagggccc

SEQ ID NO: 69 - DNA, 20 bp, synthetic construct
  misc_feature 1..20: note = None
  source 1..20: mol_type = other DNA
ggccggtatcacagggccct

SEQ ID NO: 70 - DNA, 20 bp, synthetic construct
  misc_feature 1..20: note = None
  source 1..20: mol_type = other DNA
cgcctgcctcagtttaccca

SEQ ID NO: 71 - DNA, 20 bp, synthetic construct
  misc_feature 1..20: note = None
  source 1..20: mol_type = other DNA
tcgcctgcctcagtttaccc

SEQ ID NO: 72 - DNA, 20 bp, synthetic construct
  misc_feature 1..20: note = None
  source 1..20: mol_type = other DNA
acagggccctgggtaaactg

SEQ ID NO: 73 - DNA, 20 bp, synthetic construct
  misc_feature 1..20: note = None
  source 1..20: mol_type = other DNA
ggccctgggtaaactgaggc

SEQ ID NO: 74 - DNA, 20 bp, synthetic construct
  misc_feature 1..20: note = None
  source 1..20: mol_type = other DNA
gcaggcgacacagctgcatg

SEQ ID NO: 75 - DNA, 20 bp, synthetic construct
  misc_feature 1..20: note = None
  source 1..20: mol_type = other DNA
gcgacacagctgcatgtggc

SEQ ID NO: 76 - DNA, 20 bp, synthetic construct
  misc_feature 1..20: note = None
  source 1..20: mol_type = other DNA
tacccagggccctgtgatac

SEQ ID NO: 77 - DNA, 20 bp, synthetic construct
  misc_feature 1..20: note = None
  source 1..20: mol_type = other DNA
ctgcatgtggccggtatcac

SEQ ID NO: 78 - DNA, 20 bp, synthetic construct
  misc_feature 1..20: note = None
  source 1..20: mol_type = other DNA
tgcatgtggccggtatcaca

SEQ ID NO: 79 - DNA, 20 bp, synthetic construct
  misc_feature 1..20: note = None
  source 1..20: mol_type = other DNA
tggccggtatcacagggccc

SEQ ID NO: 80 - DNA, 20 bp, synthetic construct
  misc_feature 1..20: note = None
  source 1..20: mol_type = other DNA
ggccggtatcacagggccct

SEQ ID NO: 81 - DNA, 20 bp, synthetic construct
  misc_feature 1..20: note = None
  source 1..20: mol_type = other DNA
cgcctgcctcagtttaccca

SEQ ID NO: 82 - DNA, 20 bp, synthetic construct
  misc_feature 1..20: note = None
  source 1..20: mol_type = other DNA
tcgcctgcctcagtttaccc

SEQ ID NO: 83 - DNA, 20 bp, synthetic construct
  misc_feature 1..20: note = None
  source 1..20: mol_type = other DNA
acagggccctgggtaaactg

SEQ ID NO: 84 - DNA, 20 bp, synthetic construct
  misc_feature 1..20: note = None
  source 1..20: mol_type = other DNA
ggccctgggtaaactgaggc

SEQ ID NO: 85 - DNA, 20 bp, synthetic construct
  misc_feature 1..20: note = None
  source 1..20: mol_type = other DNA
gcaggcgacacagctgcatg

SEQ ID NO: 86 - DNA, 20 bp, synthetic construct
  misc_feature 1..20: note = None
  source 1..20: mol_type = other DNA
gcgacacagctgcatgtggc

SEQ ID NO: 87 - DNA, 20 bp, synthetic construct
  misc_feature 1..20: note = None
  source 1..20: mol_type = other DNA
tacccagggccctgtgatac

SEQ ID NO: 88 - DNA, 20 bp, synthetic construct
  misc_feature 1..20: note = None
  source 1..20: mol_type = other DNA
ctgcatgtggccggtatcac

SEQ ID NO: 89 - DNA, 20 bp, synthetic construct
  misc_feature 1..20: note = None
  source 1..20: mol_type = other DNA
tgcatgtggccggtatcaca

SEQ ID NO: 90 - DNA, 20 bp, synthetic construct
  misc_feature 1..20: note = None
  source 1..20: mol_type = other DNA
tggccggtatcacagggccc

SEQ ID NO: 91 - DNA, 20 bp, synthetic construct
  misc_feature 1..20: note = None
  source 1..20: mol_type = other DNA
ggccggtatcacagggccct

SEQ ID NO: 92 - DNA, 20 bp, synthetic construct
  misc_feature 1..20: note = None
  source 1..20: mol_type = other DNA
cgcctgcctcagtttaccca

SEQ ID NO: 93 - DNA, 20 bp, synthetic construct
  misc_feature 1..20: note = None
  source 1..20: mol_type = other DNA
tcgcctgcctcagtttaccc

SEQ ID NO: 94 - DNA, 20 bp, synthetic construct
  misc_feature 1..20: note = None
  source 1..20: mol_type = other DNA
acagggccctgggtaaactg

SEQ ID NO: 95 - DNA, 20 bp, synthetic construct
  misc_feature 1..20: note = None
  source 1..20: mol_type = other DNA
ggccctgggtaaactgaggc

SEQ ID NO: 96 - DNA, 20 bp, synthetic construct
  misc_feature 1..20: note = None
  source 1..20: mol_type = other DNA
gcaggcgacacagctgcatg

SEQ ID NO: 97 - DNA, 20 bp, synthetic construct
  misc_feature 1..20: note = None
  source 1..20: mol_type = other DNA
gctgcatgtggccgtatcac

SEQ ID NO: 98 - DNA, 20 bp, synthetic construct
  misc_feature 1..20: note = None
  source 1..20: mol_type = other DNA
ttacccagggccctgtgata

SEQ ID NO: 99 - DNA, 20 bp, synthetic construct
  misc_feature 1..20: note = None
  source 1..20: mol_type = other DNA
ctgcatgtggccgtatcaca

SEQ ID NO: 100 - DNA, 20 bp, synthetic construct
  misc_feature 1..20: note = None
  source 1..20: mol_type = other DNA
gtggccgtatcacagggccc

SEQ ID NO: 101 - DNA, 20 bp, synthetic construct
  misc_feature 1..20: note = None
  source 1..20: mol_type = other DNA
tggccgtatcacagggccct

SEQ ID NO: 102 - DNA, 20 bp, synthetic construct
  misc_feature 1..20: note = None
  source 1..20: mol_type = other DNA
cacctgcctcagtttaccca

SEQ ID NO: 103 - DNA, 20 bp, synthetic construct
  misc_feature 1..20: note = None
  source 1..20: mol_type = other DNA
tcacctgcctcagtttaccc

SEQ ID NO: 104 - DNA, 20 bp, synthetic construct
  misc_feature 1..20: note = None
  source 1..20: mol_type = other DNA
acagggccctgggtaaactg

SEQ ID NO: 105 - DNA, 20 bp, synthetic construct
  misc_feature 1..20: note = None
  source 1..20: mol_type = other DNA
ggccctgggtaaactgaggc

SEQ ID NO: 106 - DNA, 20 bp, synthetic construct
  misc_feature 1..20: note = None
  source 1..20: mol_type = other DNA
gcaggtgacacagctgcatg

SEQ ID NO: 107 - DNA, 20 bp, synthetic construct
  misc_feature 1..20: note = None
  source 1..20: mol_type = other DNA
gtgacacagctgcatgtggc

SEQ ID NO: 108 - DNA, 20 bp, synthetic construct
  misc_feature 1..20: note = None
  source 1..20: mol_type = other DNA
gctgcatgtggccggtatca

SEQ ID NO: 109 - DNA, 20 bp, synthetic construct
  misc_feature 1..20: note = None
  source 1..20: mol_type = other DNA
tatccagggccccgtgatac

SEQ ID NO: 110 - DNA, 20 bp, synthetic construct
  misc_feature 1..20: note = None
  source 1..20: mol_type = other DNA
ctgcatgtggccggtatcac

SEQ ID NO: 111 - DNA, 20 bp, synthetic construct
  misc_feature 1..20: note = None
  source 1..20: mol_type = other DNA
tgcatgtggccggtatcacg

SEQ ID NO: 112 - DNA, 20 bp, synthetic construct
  misc_feature 1..20: note = None
  source 1..20: mol_type = other DNA
tggccggtatcacggggccc

SEQ ID NO: 113 - DNA, 20 bp, synthetic construct
  misc_feature 1..20: note = None
  source 1..20: mol_type = other DNA
cgcctgcctctgtttatcca

SEQ ID NO: 114 - DNA, 20 bp, synthetic construct
  misc_feature 1..20: note = None
  source 1..20: mol_type = other DNA
tcgcctgcctctgtttatcc

SEQ ID NO: 115 - DNA, 20 bp, synthetic construct
  misc_feature 1..20: note = None
  source 1..20: mol_type = other DNA
acggggccctggataaacag

SEQ ID NO: 116 - DNA, 20 bp, synthetic construct
  misc_feature 1..20: note = None
  source 1..20: mol_type = other DNA
ggccctggataaacagaggc

SEQ ID NO: 117 - DNA, 20 bp, synthetic construct
  misc_feature 1..20: note = None
  source 1..20: mol_type = other DNA
gcaggcgacacagctgcatg

SEQ ID NO: 118 - DNA, 20 bp, synthetic construct
  misc_feature 1..20: note = None
  source 1..20: mol_type = other DNA
gcgacacagctgcatgtggc

SEQ ID NO: 119 - DNA, 20 bp, synthetic construct
  misc_feature 1..20: note = None
  source 1..20: mol_type = other DNA
gctgcatgtggccggtatca

SEQ ID NO: 120 - DNA, 20 bp, synthetic construct
  misc_feature 1..20: note = None
  source 1..20: mol_type = other DNA
tacccagggccccgtgatac

SEQ ID NO: 121 - DNA, 20 bp, synthetic construct
  misc_feature 1..20: note = None
  source 1..20: mol_type = other DNA
ctgcatgtggccggtatcac

SEQ ID NO: 122 - DNA, 20 bp, synthetic construct
  misc_feature 1..20: note = None
  source 1..20: mol_type = other DNA
tgcatgtggccggtatcacg

SEQ ID NO: 123 - DNA, 20 bp, synthetic construct
  misc_feature 1..20: note = None
  source 1..20: mol_type = other DNA
tggccggtatcacggggccc

SEQ ID NO: 124 - DNA, 20 bp, synthetic construct
  misc_feature 1..20: note = None
  source 1..20: mol_type = other DNA
ggccggtatcacggggccct

SEQ ID NO: 125 - DNA, 20 bp, synthetic construct
  misc_feature 1..20: note = None
  source 1..20: mol_type = other DNA
cgcctgcctcagtttaccca

SEQ ID NO: 126 - DNA, 20 bp, synthetic construct
  misc_feature 1..20: note = None
  source 1..20: mol_type = other DNA
tcgcctgcctcagtttaccc

SEQ ID NO: 127 - DNA, 20 bp, synthetic construct
  misc_feature 1..20: note = None
  source 1..20: mol_type = other DNA
acggggccctgggtaaactg

SEQ ID NO: 128 - DNA, 20 bp, synthetic construct
  misc_feature 1..20: note = None
  source 1..20: mol_type = other DNA
ggccctgggtaaactgaggc

SEQ ID NO: 129 - DNA, 20 bp, synthetic construct
  misc_feature 1..20: note = None
  source 1..20: mol_type = other DNA
tgggtaaactgaggcaggcg

SEQ ID NO: 130 - DNA, 20 bp, synthetic construct
  misc_feature 1..20: note = None
  source 1..20: mol_type = other DNA
cctgagggacttgatggggg

SEQ ID NO: 131 - DNA, 20 bp, synthetic construct
  misc_feature 1..20: note = None
  source 1..20: mol_type = other DNA
agacctgagggacttgatgg

SEQ ID NO: 132 - DNA, 20 bp, synthetic construct
  misc_feature 1..20: note = None
  source 1..20: mol_type = other DNA
tagacctgagggacttgatg

SEQ ID NO: 133 - DNA, 20 bp, synthetic construct
  misc_feature 1..20: note = None
  source 1..20: mol_type = other DNA
ctagacctgagggacttgat

SEQ ID NO: 134 - DNA, 20 bp, synthetic construct
  misc_feature 1..20: note = None
  source 1..20: mol_type = other DNA
cctagacctgagggacttga

SEQ ID NO: 135 - DNA, 20 bp, synthetic construct
  misc_feature 1..20: note = None
  source 1..20: mol_type = other DNA
ccacccccatcaagtccctc

SEQ ID NO: 136 - DNA, 20 bp, synthetic construct
  misc_feature 1..20: note = None
  source 1..20: mol_type = other DNA
cctgccaaacctagacctga

SEQ ID NO: 137 - DNA, 20 bp, synthetic construct
  misc_feature 1..20: note = None
  source 1..20: mol_type = other DNA
acctgccaaacctagacctg

SEQ ID NO: 138 - DNA, 20 bp, synthetic construct
  misc_feature 1..20: note = None
  source 1..20: mol_type = other DNA
ccatcaagtccctcaggtct

SEQ ID NO: 139 - DNA, 20 bp, synthetic construct
  misc_feature 1..20: note = None
  source 1..20: mol_type = other DNA
aagtccctcaggtctaggtt

SEQ ID NO: 140 - DNA, 20 bp, synthetic construct
  misc_feature 1..20: note = None
  source 1..20: mol_type = other DNA
ccctcaggtctaggtttggc

SEQ ID NO: 141 - DNA, 20 bp, synthetic construct
  misc_feature 1..20: note = None
  source 1..20: mol_type = other DNA
aggtctaggtttggcaggtt

SEQ ID NO: 142 - DNA, 20 bp, synthetic construct
  misc_feature 1..20: note = None
  source 1..20: mol_type = other DNA
ggcaaaaacacagcaacgct

SEQ ID NO: 143 - DNA, 20 bp, synthetic construct
  misc_feature 1..20: note = None
  source 1..20: mol_type = other DNA
gctcggttaaatctgaattt

SEQ ID NO: 144 - DNA, 20 bp, synthetic construct
  misc_feature 1..20: note = None
  source 1..20: mol_type = other DNA
ctcggttaaatctgaatttc

SEQ ID NO: 145 - DNA, 20 bp, synthetic construct
  misc_feature 1..20: note = None
  source 1..20: mol_type = other DNA
aatttcgggtaagtatatcc

SEQ ID NO: 146 - DNA, 20 bp, synthetic construct
  misc_feature 1..20: note = None
  source 1..20: mol_type = other DNA
atttcgggtaagtatatcct

SEQ ID NO: 147 - DNA, 20 bp, synthetic construct
  misc_feature 1..20: note = None
  source 1..20: mol_type = other DNA
gtctcttccaaatgaggccc

SEQ ID NO: 148 - DNA, 20 bp, synthetic construct
  misc_feature 1..20: note = None
  source 1..20: mol_type = other DNA
agtatatcctgggcctcatt

SEQ ID NO: 149 - DNA, 20 bp, synthetic construct
  misc_feature 1..20: note = None
  source 1..20: mol_type = other DNA
atctaagtctcttccaaatg

SEQ ID NO: 150 - DNA, 20 bp, synthetic construct
  misc_feature 1..20: note = None
  source 1..20: mol_type = other DNA
aaaaaacgtcgagaccagcc

SEQ ID NO: 151 - DNA, 20 bp, synthetic construct
  misc_feature 1..20: note = None
  source 1..20: mol_type = other DNA
tttcaccgtgttggccgggc

SEQ ID NO: 152 - DNA, 20 bp, synthetic construct
  misc_feature 1..20: note = None
  source 1..20: mol_type = other DNA
ggggtttcaccgtgttggcc

SEQ ID NO: 153 - DNA, 20 bp, synthetic construct
  misc_feature 1..20: note = None
  source 1..20: mol_type = other DNA
cggggtttcaccgtgttggc

SEQ ID NO: 154 - DNA, 20 bp, synthetic construct
  misc_feature 1..20: note = None
  source 1..20: mol_type = other DNA
cgagaccagcccggccaaca

SEQ ID NO: 155 - DNA, 20 bp, synthetic construct
  misc_feature 1..20: note = None
  source 1..20: mol_type = other DNA
gagacggggtttcaccgtgt

SEQ ID NO: 156 - DNA, 20 bp, synthetic construct
  misc_feature 1..20: note = None
  source 1..20: mol_type = other DNA
ttgtatttttagtagagacg

SEQ ID NO: 157 - DNA, 20 bp, synthetic construct
  misc_feature 1..20: note = None
  source 1..20: mol_type = other DNA
tttgtatttttagtagagac

SEQ ID NO: 158 - DNA, 20 bp, synthetic construct
  misc_feature 1..20: note = None
  source 1..20: mol_type = other DNA
ttttgtatttttagtagaga

SEQ ID NO: 159 - DNA, 20 bp, synthetic construct
  misc_feature 1..20: note = None
  source 1..20: mol_type = other DNA
aaaaatacaaaaaattagcc

SEQ ID NO: 160 - DNA, 20 bp, synthetic construct
  misc_feature 1..20: note = None
  source 1..20: mol_type = other DNA
caggcgtgagccactgcgcc

SEQ ID NO: 161 - DNA, 20 bp, synthetic construct
  misc_feature 1..20: note = None
  source 1..20: mol_type = other DNA
aaaaaattagccaggcgcag

SEQ ID NO: 162 - DNA, 20 bp, synthetic construct
  misc_feature 1..20: note = None
  source 1..20: mol_type = other DNA
tcccagagtgctgggatcac

SEQ ID NO: 163 - DNA, 20 bp, synthetic construct
  misc_feature 1..20: note = None
  source 1..20: mol_type = other DNA
cctcagcctcccagagtgct

SEQ ID NO: 164 - DNA, 20 bp, synthetic construct
  misc_feature 1..20: note = None
  source 1..20: mol_type = other DNA
cgcctgtgatcccagcactc

SEQ ID NO: 165 - DNA, 20 bp, synthetic construct
  misc_feature 1..20: note = None
  source 1..20: mol_type = other DNA
gcctcagcctcccagagtgc

SEQ ID NO: 166 - DNA, 20 bp, synthetic construct
  misc_feature 1..20: note = None
  source 1..20: mol_type = other DNA
gcctgtgatcccagcactct

SEQ ID NO: 167 - DNA, 20 bp, synthetic construct
  misc_feature 1..20: note = None
  source 1..20: mol_type = other DNA
tgtgatcccagcactctggg

SEQ ID NO: 168 - DNA, 20 bp, synthetic construct
  misc_feature 1..20: note = None
  source 1..20: mol_type = other DNA
cccagcactctgggaggctg

SEQ ID NO: 169 - DNA, 20 bp, synthetic construct
  misc_feature 1..20: note = None
  source 1..20: mol_type = other DNA
gcactctgggaggctgaggc

SEQ ID NO: 170 - DNA, 20 bp, synthetic construct
  misc_feature 1..20: note = None
  source 1..20: mol_type = other DNA
ctctgggaggctgaggcagg

SEQ ID NO: 171 - DNA, 20 bp, synthetic construct
  misc_feature 1..20: note = None
  source 1..20: mol_type = other DNA
tgaggcaggcggatcacccg

SEQ ID NO: 172 - DNA, 20 bp, synthetic construct
  misc_feature 1..20: note = None
  source 1..20: mol_type = other DNA
ggtcttgaacatctgacctc

SEQ ID NO: 173 - DNA, 20 bp, synthetic construct
  misc_feature 1..20: note = None
  source 1..20: mol_type = other DNA
tggtcttgaacatctgacct

SEQ ID NO: 174 - DNA, 20 bp, synthetic construct
  misc_feature 1..20: note = None
  source 1..20: mol_type = other DNA
tcagatgttcaagaccagcc

SEQ ID NO: 175 - DNA, 20 bp, synthetic construct
  misc_feature 1..20: note = None
  source 1..20: mol_type = other DNA
tttcgccctgtcggccaggc

SEQ ID NO: 176 - DNA, 20 bp, synthetic construct
  misc_feature 1..20: note = None
  source 1..20: mol_type = other DNA
agtgtttcgccctgtcggcc

SEQ ID NO: 177 - DNA, 20 bp, synthetic construct
  misc_feature 1..20: note = None
  source 1..20: mol_type = other DNA
tcaagaccagcctggccgac

SEQ ID NO: 178 - DNA, 20 bp, synthetic construct
  misc_feature 1..20: note = None
  source 1..20: mol_type = other DNA
caagaccagcctggccgaca

SEQ ID NO: 179 - DNA, 20 bp, synthetic construct
  misc_feature 1..20: note = None
  source 1..20: mol_type = other DNA
gagacagtgtttcgccctgt

SEQ ID NO: 180 - DNA, 20 bp, synthetic construct
  misc_feature 1..20: note = None
  source 1..20: mol_type = other DNA
ctacaaatacaaaaattagc

SEQ ID NO: 181 - DNA, 20 bp, synthetic construct
  misc_feature 1..20: note = None
  source 1..20: mol_type = other DNA
tacaaatacaaaaattagcc

SEQ ID NO: 182 - DNA, 20 bp, synthetic construct
  misc_feature 1..20: note = None
  source 1..20: mol_type = other DNA
atacaaaaattagccgggag

SEQ ID NO: 183 - DNA, 20 bp, synthetic construct
  misc_feature 1..20: note = None
  source 1..20: mol_type = other DNA
caggcacctgccaccactcc

SEQ ID NO: 184 - DNA, 20 bp, synthetic construct
  misc_feature 1..20: note = None
  source 1..20: mol_type = other DNA
caaaaattagccgggagtgg

SEQ ID NO: 185 - DNA, 20 bp, synthetic construct
  misc_feature 1..20: note = None
  source 1..20: mol_type = other DNA
aattagccgggagtggtggc

SEQ ID NO: 186 - DNA, 20 bp, synthetic construct
  misc_feature 1..20: note = None
  source 1..20: mol_type = other DNA
tcctgaatagctgagattac

SEQ ID NO: 187 - DNA, 20 bp, synthetic construct
  misc_feature 1..20: note = None
  source 1..20: mol_type = other DNA
gcctgtaatctcagctattc

SEQ ID NO: 188 - DNA, 20 bp, synthetic construct
  misc_feature 1..20: note = None
  source 1..20: mol_type = other DNA
tgtaatctcagctattcagg

SEQ ID NO: 189 - DNA, 20 bp, synthetic construct
  misc_feature 1..20: note = None
  source 1..20: mol_type = other DNA
ctcagctattcaggaggctg

SEQ ID NO: 190 - DNA, 20 bp, synthetic construct
  misc_feature 1..20: note = None
  source 1..20: mol_type = other DNA
gctattcaggaggctgaggc

SEQ ID NO: 191 - DNA, 20 bp, synthetic construct
  misc_feature 1..20: note = None
  source 1..20: mol_type = other DNA
gcaggagaatcacttgaacc

SEQ ID NO: 192 - DNA, 20 bp, synthetic construct
  misc_feature 1..20: note = None
  source 1..20: mol_type = other DNA
caggagaatcacttgaacct

SEQ ID NO: 193 - DNA, 20 bp, synthetic construct
  misc_feature 1..20: note = None
  source 1..20: mol_type = other DNA
gagaatcacttgaacctggg

SEQ ID NO: 194 - DNA, 20 bp, synthetic construct
  misc_feature 1..20: note = None
  source 1..20: mol_type = other DNA
aatcacttgaacctgggagg

SEQ ID NO: 195 - DNA, 20 bp, synthetic construct
  misc_feature 1..20: note = None
  source 1..20: mol_type = other DNA
cacggcaacctccgcctccc

SEQ ID NO: 196 - DNA, 20 bp, synthetic construct
  misc_feature 1..20: note = None
  source 1..20: mol_type = other DNA
cacttgaacctgggaggcgg

SEQ ID NO: 197 - DNA, 20 bp, synthetic construct
  misc_feature 1..20: note = None
  source 1..20: mol_type = other DNA
aggcggaggttgccgtgagc

SEQ ID NO: 198 - DNA, 20 bp, synthetic construct
  misc_feature 1..20: note = None
  source 1..20: mol_type = other DNA
ggcggaggttgccgtgagcc

SEQ ID NO: 199 - DNA, 20 bp, synthetic construct
  misc_feature 1..20: note = None
  source 1..20: mol_type = other DNA
ggtggcgtgatcccggctca

SEQ ID NO: 200 - DNA, 20 bp, synthetic construct
  misc_feature 1..20: note = None
  source 1..20: mol_type = other DNA
ggagtgcggtggcgtgatcc

SEQ ID NO: 201 - DNA, 20 bp, synthetic construct
  misc_feature 1..20: note = None
  source 1..20: mol_type = other DNA
tcgcccaggctggagtgcgg

SEQ ID NO: 202 - DNA, 20 bp, synthetic construct
  misc_feature 1..20: note = None
  source 1..20: mol_type = other DNA
ctatcgcccaggctggagtg

SEQ ID NO: 203 - DNA, 20 bp, synthetic construct
  misc_feature 1..20: note = None
  source 1..20: mol_type = other DNA
cacgccaccgcactccagcc

SEQ ID NO: 204 - DNA, 20 bp, synthetic construct
  misc_feature 1..20: note = None
  source 1..20: mol_type = other DNA
acgccaccgcactccagcct

SEQ ID NO: 205 - DNA, 20 bp, synthetic construct
  misc_feature 1..20: note = None
  source 1..20: mol_type = other DNA
tcttgctctatcgcccaggc

SEQ ID NO: 206 - DNA, 20 bp, synthetic construct
  misc_feature 1..20: note = None
  source 1..20: mol_type = other DNA
agagtcttgctctatcgccc

SEQ ID NO: 207 - DNA, 20 bp, synthetic construct
  misc_feature 1..20: note = None
  source 1..20: mol_type = other DNA
ggttttttaatttatttttt

SEQ ID NO: 208 - DNA, 20 bp, synthetic construct
  misc_feature 1..20: note = None
  source 1..20: mol_type = other DNA
aaatgtcagataatcaatgt

SEQ ID NO: 209 - DNA, 20 bp, synthetic construct
  misc_feature 1..20: note = None
  source 1..20: mol_type = other DNA
caaatgtcagataatcaatg

SEQ ID NO: 210 - DNA, 20 bp, synthetic construct
  misc_feature 1..20: note = None
  source 1..20: mol_type = other DNA
ggggcctccagacaaaattc

SEQ ID NO: 211 - DNA, 20 bp, synthetic construct
  misc_feature 1..20: note = None
  source 1..20: mol_type = other DNA
tgtgcatcctgaattttgtc

SEQ ID NO: 212 - DNA, 20 bp, synthetic construct
  misc_feature 1..20: note = None
  source 1..20: mol_type = other DNA
gcatcctgaattttgtctgg

SEQ ID NO: 213 - DNA, 20 bp, synthetic construct
  misc_feature 1..20: note = None
  source 1..20: mol_type = other DNA
acgctggattggctcgggtg

SEQ ID NO: 214 - DNA, 20 bp, synthetic construct
  misc_feature 1..20: note = None
  source 1..20: mol_type = other DNA
gacgctggattggctcgggt

SEQ ID NO: 215 - DNA, 20 bp, synthetic construct
  misc_feature 1..20: note = None
  source 1..20: mol_type = other DNA
agacgctggattggctcggg

SEQ ID NO: 216 - DNA, 20 bp, synthetic construct
  misc_feature 1..20: note = None
  source 1..20: mol_type = other DNA
acaagacgctggattggctc

SEQ ID NO: 217 - DNA, 20 bp, synthetic construct
  misc_feature 1..20: note = None
  source 1..20: mol_type = other DNA
gacaagacgctggattggct

SEQ ID NO: 218 - DNA, 20 bp, synthetic construct
  misc_feature 1..20: note = None
  source 1..20: mol_type = other DNA
agggggacaagacgctggat

SEQ ID NO: 219 - DNA, 20 bp, synthetic construct
  misc_feature 1..20: note = None
  source 1..20: mol_type = other DNA
ggagaagggggacaagacgc

SEQ ID NO: 220 - DNA, 20 bp, synthetic construct
  misc_feature 1..20: note = None
  source 1..20: mol_type = other DNA
tgatgaaaagggggagaagg

SEQ ID NO: 221 - DNA, 20 bp, synthetic construct
  misc_feature 1..20: note = None
  source 1..20: mol_type = other DNA
ttgatgaaaagggggagaag

SEQ ID NO: 222 - DNA, 20 bp, synthetic construct
  misc_feature 1..20: note = None
  source 1..20: mol_type = other DNA
gttgatgaaaagggggagaa

SEQ ID NO: 223 - DNA, 20 bp, synthetic construct
  misc_feature 1..20: note = None
  source 1..20: mol_type = other DNA
cgttgatgaaaagggggaga

SEQ ID NO: 224 - DNA, 20 bp, synthetic construct
  misc_feature 1..20: note = None
  source 1..20: mol_type = other DNA
acagggcgttgatgaaaagg

SEQ ID NO: 225 - DNA, 20 bp, synthetic construct
  misc_feature 1..20: note = None
  source 1..20: mol_type = other DNA
cacagggcgttgatgaaaag

SEQ ID NO: 226 - DNA, 20 bp, synthetic construct
  misc_feature 1..20: note = None
  source 1..20: mol_type = other DNA
gcacagggcgttgatgaaaa

SEQ ID NO: 227 - DNA, 20 bp, synthetic construct
  misc_feature 1..20: note = None
  source 1..20: mol_type = other DNA
ggcacagggcgttgatgaaa

SEQ ID NO: 228 - DNA, 20 bp, synthetic construct
  misc_feature 1..20: note = None
  source 1..20: mol_type = other DNA
tttcatcaacgccctgtgcc

SEQ ID NO: 229 - DNA, 20 bp, synthetic construct
  misc_feature 1..20: note = None
  source 1..20: mol_type = other DNA
acttcctctcccctggcaca

SEQ ID NO: 230 - DNA, 20 bp, synthetic construct
  misc_feature 1..20: note = None
  source 1..20: mol_type = other DNA
ttcatcaacgccctgtgcca

SEQ ID NO: 231 - DNA, 20 bp, synthetic construct
  misc_feature 1..20: note = None
  source 1..20: mol_type = other DNA
cacttcctctcccctggcac

SEQ ID NO: 232 - DNA, 20 bp, synthetic construct
  misc_feature 1..20: note = None
  source 1..20: mol_type = other DNA
tcatcaacgccctgtgccag

SEQ ID NO: 233 - DNA, 20 bp, synthetic construct
  misc_feature 1..20: note = None
  source 1..20: mol_type = other DNA
aacgccctgtgccaggggag

SEQ ID NO: 234 - DNA, 20 bp, synthetic construct
  misc_feature 1..20: note = None
  source 1..20: mol_type = other DNA
gccctccacttcctctcccc

SEQ ID NO: 235 - DNA, 20 bp, synthetic construct
  misc_feature 1..20: note = None
  source 1..20: mol_type = other DNA
ctgtgccaggggagaggaag

SEQ ID NO: 236 - DNA, 20 bp, synthetic construct
  misc_feature 1..20: note = None
  source 1..20: mol_type = other DNA
tgccaggggagaggaagtgg

SEQ ID NO: 237 - DNA, 20 bp, synthetic construct
  misc_feature 1..20: note = None
  source 1..20: mol_type = other DNA
gccaggggagaggaagtgga

SEQ ID NO: 238 - DNA, 20 bp, synthetic construct
  misc_feature 1..20: note = None
  source 1..20: mol_type = other DNA
ggagaggaagtggagggcgc

SEQ ID NO: 239 - DNA, 20 bp, synthetic construct
  misc_feature 1..20: note = None
  source 1..20: mol_type = other DNA
aggaagtggagggcgctggc

SEQ ID NO: 240 - DNA, 20 bp, synthetic construct
  misc_feature 1..20: note = None
  source 1..20: mol_type = other DNA
tggagggcgctggccggccg

SEQ ID NO: 241 - DNA, 20 bp, synthetic construct
  misc_feature 1..20: note = None
  source 1..20: mol_type = other DNA
ggagggcgctggccggccgt

SEQ ID NO: 242 - DNA, 20 bp, synthetic construct
  misc_feature 1..20: note = None
  source 1..20: mol_type = other DNA
gagggcgctggccggccgtg

SEQ ID NO: 243 - DNA, 20 bp, synthetic construct
  misc_feature 1..20: note = None
  source 1..20: mol_type = other DNA
ccgttgcattgccccacggc

SEQ ID NO: 244 - DNA, 20 bp, synthetic construct
  misc_feature 1..20: note = None
  source 1..20: mol_type = other DNA
ccggccgtggggcaatgcaa

SEQ ID NO: 245 - DNA, 20 bp, synthetic construct
  misc_feature 1..20: note = None
  source 1..20: mol_type = other DNA
tcttatagccctgtgctggg

SEQ ID NO: 246 - DNA, 20 bp, synthetic construct
  misc_feature 1..20: note = None
  source 1..20: mol_type = other DNA
atgcaacggcctcccagcac

SEQ ID NO: 247 - DNA, 20 bp, synthetic construct
  misc_feature 1..20: note = None
  source 1..20: mol_type = other DNA
tgcaacggcctcccagcaca

SEQ ID NO: 248 - DNA, 20 bp, synthetic construct
  misc_feature 1..20: note = None
  source 1..20: mol_type = other DNA
tcctcttatagccctgtgct

SEQ ID NO: 249 - DNA, 20 bp, synthetic construct
  misc_feature 1..20: note = None
  source 1..20: mol_type = other DNA
ctcctcttatagccctgtgc

SEQ ID NO: 250 - DNA, 20 bp, synthetic construct
  misc_feature 1..20: note = None
  source 1..20: mol_type = other DNA
tcccagcacagggctataag

SEQ ID NO: 251 - DNA, 20 bp, synthetic construct
  misc_feature 1..20: note = None
  source 1..20: mol_type = other DNA
cacagggctataagaggagc

SEQ ID NO: 252 - DNA, 20 bp, synthetic construct
  misc_feature 1..20: note = None
  source 1..20: mol_type = other DNA
acagggctataagaggagcc

SEQ ID NO: 253 - DNA, 20 bp, synthetic construct
  misc_feature 1..20: note = None
  source 1..20: mol_type = other DNA
gtgtcwtcctcgcctgtgtc

SEQ ID NO: 254 - DNA, 20 bp, synthetic construct
  misc_feature 1..20: note = None
  source 1..20: mol_type = other DNA
cccacaatctccgaggccag

SEQ ID NO: 255 - DNA, 19 bp, synthetic construct
  misc_feature 1..19: note = None
  source 1..19: mol_type = other DNA
ctgggctacactgagcacc

SEQ ID NO: 256 - DNA, 21 bp, synthetic construct
  misc_feature 1..21: note = None
  source 1..21: mol_type = other DNA
aagtggtcgttgagggcaatg

SEQ ID NO: 257 - DNA, 21 bp, synthetic construct
  misc_feature 1..21: note = None
  source 1..21: mol_type = other DNA
catgtacgttgctatccaggc

SEQ ID NO: 258 - DNA, 21 bp, synthetic construct
  misc_feature 1..21: note = None
  source 1..21: mol_type = other DNA
ctccttaatgtcacgcacgat

SEQ ID NO: 259 - DNA, 149 bp, synthetic construct
  misc_feature 1..149: note = None
  source 1..149: mol_type = other DNA
tgccaggggagaggaagtggagggcgctggccggccgtggggcaatgcaacggcctcccagcacagggctataagaggagccgggcgggcacggaggggcagagaccccggagccccagccccaccatgaccctcggccgccgactcgc

SEQ ID NO: 260 - DNA, 23 bp, synthetic construct
  unsure 21: note = None
  source 1..23: mol_type = other DNA
gaggccgttgcattgccccangg

SEQ ID NO: 261 - DNA, 23 bp, synthetic construct
  unsure 21: note = None
  source 1..23: mol_type = other DNA
gggctataagaggagccgggngg

SEQ ID NO: 262 - DNA, 34 bp, synthetic construct
  misc_feature 1..34: note = None
  source 1..34: mol_type = other DNA
gtttaattgagttgtcatatgttaataacggtat

SEQ ID NO: 263 - DNA, 34 bp, synthetic construct
  misc_feature 1..34: note = None
  source 1..34: mol_type = other DNA
ataccgttattaacatatgacaactcaattaaac

(19) United States
(12) Patent Application Publication
(10) Pub. No.: US 2024/0018477 A1
(43) Pub. Date: Jan. 18, 2024

(54) METHOD AND COMPOSITION FOR A TARGETED GENE KNOCKOUT

(71) Applicant: Eberhard Karls Universität Tübingen Medizinische Fakultät, Tübingen (DE)

(72) Inventors: Julia Skokowa, Kusterdingen (DE); Masoud Nasri, Tübingen (DE); Perihan Mir, Leinfelden-Echterdingen (DE)

(21) Appl. No.: 18/453,993

(22) Filed: Aug. 22, 2023

Related U.S. Application Data
(63) Continuation of application No. PCT/EP2022/053273, filed on Feb. 10, 2022.

(30) Foreign Application Priority Data
Feb. 22, 2021 (EP) 21158366.1

(51) Int. Cl. C12N 5/0789; C12N 15/113; A61P 37/02

(52) U.S. Cl. CPC C12N 5/0647; C12N 15/1137; A61P 37/02; C12N 2310/20; C12Y 304/21037

(57) ABSTRACT

A composition is for the targeted knockout of a gene on double-stranded DNA in a biological cell. A method is for the targeted knockout of a gene on double-stranded DNA in a biological cell. A preparation includes a biological cell prepared in vitro. The biological cell includes a gene on double-stranded DNA, which is knocked-out in a targeted manner. A kit is for the targeted knockout of a gene on double-stranded DNA in a biological cell. Another method is for treating a subject afflicted with a disease associated with a mutated gene. Nucleic acid molecules can be a component of the composition and methods.

### CROSS-REFERENCES TO RELATED APPLICATIONS

This application is a continuation of copending International Patent Application PCT/EP2022/053273 filed on 10 Feb. 2022 and designating the United States of America, which was published under PCT Article 21(2) in English, and claims priority of European patent application EP 21 158 366.1 filed on 22 Feb. 2021, all of which are incorporated herein by reference in their entirety.

### FIELD OF THE INVENTION

The present invention is directed to a composition for the targeted knockout of a gene on double-stranded DNA in a biological cell, a method for the targeted knockout of a gene on double-stranded DNA in a biological cell, a preparation comprising a biological cell prepared in vitro, said biological cell comprises a gene on double-stranded DNA, which is knocked-out in a targeted manner, a kit for the targeted knockout of a gene on double-stranded DNA in a biological cell, a method of treating a subject afflicted with a disease associated with a mutated gene, nucleic acid molecules which can be a component of said composition and methods, and a method of treating a subject afflicted with a disease associated with a mutated gene.

### REFERENCE TO ELECTRONIC SEQUENCE LISTING

The present application is filed along with an Electronic Sequence Listing. The Electronic Sequence Listing is provided as a file entitled WWELL014.013C1_SEQ_LIST.xml, which is approximately 322 kilobytes in size, created on Aug. 22, 2023. The information in the Electronic Sequence Listing is incorporated herein by reference in its entirety.

The present invention relates to the field of molecular medicine, more particularly to the field of genetic engineering applications, preferably to the targeted knockout of disease-associated genes.

### BACKGROUND OF THE INVENTION

A genetic disorder is a health problem caused by one or more abnormalities in the genome. It can be caused by a mutation in a single gene (monogenic) or multiple genes (polygenic) or by a chromosomal abnormality. Although polygenic disorders are the most common, the term is mostly used when discussing disorders with a single genetic cause, either in a gene or chromosome.

Two copies of the gene must be mutated for a person to be affected by an autosomal recessive disorder. An affected person usually has unaffected parents who each carry a single copy of the mutated gene and are referred to as genetic carriers. Each parent with a defective gene normally do not have symptoms. Two unaffected people who each carry one copy of the mutated gene have a 25% risk with each pregnancy of having a child affected by the disorder.

Examples of autosomal recessive disorders are albinism, medium-chain acyl-CoA dehydrogenase deficiency, cystic fibrosis, sickle cell disease, Tay-Sachs disease, Niemann-Pick disease, spinal muscular atrophy, and Roberts syndrome.

Only one mutated copy of the gene will be necessary for a person to be affected by an autosomal dominant disorder. Each affected person usually has one affected parent. The chance a child will inherit the mutated gene is 50%. Autosomal dominant conditions sometimes have reduced penetrance, which means although only one mutated copy is needed, not all individuals who inherit that mutation go on to develop the disease.

Examples of autosomal dominant disorders are Huntington's disease, neurofibromatosis type 1, neurofibromatosis type 2, Marfan syndrome, hereditary nonpolyposis colorectal cancer, hereditary multiple exostoses (a highly penetrant autosomal dominant disorder), tuberous sclerosis, Von Willebrand disease, and acute intermittent porphyria. Birth defects are also called congenital anomalies.

Neutropenia is an abnormally low concentration of neutrophils in the blood. Neutrophils make up the majority of circulating white blood cells and serve as the primary defense against infections by destroying bacteria, bacterial fragments and immunoglobulin-bound viruses in the blood. People with neutropenia are more susceptible to bacterial infections and, without prompt medical attention, the condition may become life-threatening. Neutropenia can be divided into congenital and acquired, with severe congenital neutropenia and cyclic neutropenia.

Severe congenital neutropenia (SCN or CN), also often known as Kostmann syndrome or disease, is an inherited bone marrow failure syndrome. CN affects myelopoiesis, usually without other physical malformations.

Autosomal-dominant mutations in the so-called “elastase neutrophil expressed gene” (ELANE) are the most common cause of CN (ELANE-CN). “Maturation arrest”, the failure of the bone marrow myeloid progenitors to form mature neutrophils, is a consistent feature of ELANE-CN. Mutated neutrophil elastase could not be properly processed, accumulates in the cells, inducing unfolded protein response (UPR) and endoplasmic reticulum (ER) stress. This leads to the death of hematopoietic cells and defective granulopoiesis. ELANE-CN manifests in infancy with life-threatening bacterial infections.

Another congenital bone marrow failure syndrome which is caused by ELANE mutations is cyclic neutropenia (ELANE-CyN). In CyN patients the numbers of mature neutrophils show cycling from zero to almost normal levels (under G-CSF treatment) in approximately 21-days intervals with a high probability of severe infections during the neutropenia phase.

Daily administration of granulocyte colony stimulating factor (G-CSF), mostly in recombinant form or the human variant (rhG-CSF; filgrastim), to patients suffering from ELANE-CN and ELANE-CyN in many cases clinically improves neutrophil counts and immune function. G-CSF stimulates the production of more neutrophils and delays their apoptosis. It often decreases the severity and frequency of infections and, therefore, is the mainstay of therapy. Overall survival is now estimated to exceed 80% although 10% of the patients still die from severe bacterial infections or sepsis.

This form of G-CSF therapy of ELANE-CN and ELANE-CyN may, however, increase the risk for long term side effects. Approximately 20% of the patients develop myelodysplasia or acute myeloid leukemia. The most common leukemia in CN and CyN is AML, but acute lymphoid leukemia (ALL), juvenile myelomonocytic leukemia (JMML), chronic myelomonocytic leukemia (CMML), and bi-phenotypic leukemia are also reported in the literature. It was previously demonstrated that patients who had a robust response to G-CSF (doses <8 pg/kg/day) had a cumulative incidence of 15% for developing MDS/leukemia after 15 years on G-CSF, while an incidence of 34% was reported in patients with poor response to G-CSF despite high doses. In addition, approximately 15% of CN and CyN patients do not respond to G-CSF even at doses of up to 50 μg/kg/day.

Hematopoietic stem cell transplant (HSCT) is an alternative, curative therapy for patients who do not respond to G-CSF therapy or who develop AML/MDS. However, patients with chronic neutropenia who undergo HCT are at increased risk of developing infectious complications such as fungal and graft-versus-host disease. Moreover, HCT requires a matched related donor for successful survival but most patients will not have an available matched donor.

WO 2019/217294 discloses a method for inactivating a mutant allele of the ELANE gene associated with CN and CyN. The known method involves the introduction to a biological cell of a CRISPR nuclease or a sequence encoding the CRISPR nuclease together with a guide RNA molecule. The complex of the CRISPR nuclease and the RNA molecule is described to affect a double strand break in the mutant allele of the ELANE gene. This method is complex and cumbersome and has to be adapted to each patient individually. It depends on the existence of single nucleotide polymorphisms (SNPs) which, however, cannot be found or used in all patients in the same manner.

Nasri et al. (2020), CRISPR/Cas9-mediated ELANE knockout enables neutrophilic maturation of primary hematopoietic stem and progenitor cells and induced pluripotent stem cells of severe congenital neutropenia patients, Haematologica 105(3), pp. 598-609, disclose a similar method. In primary hematopoietic stem and progenitor cells from ELANE-CN and ELANE-CyN patients the mutated ELANE gene is knocked-out by causing DNA double-strand breaks by introducing a CRISPR/Cas9-sgRNA ribonucleo-protein.

However, the approaches disclosed in WO 2019/27294 and Nasri et al. may result in the introduction of novel ELANE mutations and malignant transformations of the treated cells and, for this reason, may be inappropriate for an application in clinical practices.

### SUMMARY OF THE INVENTION

Against this background it is an object underlying the invention to provide an improved method and composition for the targeted knockout of a gene on double-stranded DNA in a biological cell, by means of which the disadvantages of the methods known in the art can be avoided or at least reduced. In, particular a method and composition should be provided allowing the treatment of an autosomal-dominantly inherited genetic disorder, such as congenital neutropenia (CN) or cyclic neutropenia (CyN), in particular ELANE-CN and ELANE-CyN, in a reliable and safe manner.

The present invention satisfies these and other needs.

The present invention provides a composition for the targeted knockout of a gene on double-stranded DNA in a biological cell, comprising:
a first CRISPR endonuclease or a nucleic acid molecule encoding the said CRISPR endonuclease:
a second CRISPR endonuclease or a nucleic acid molecule encoding said second CRISPR endonuclease;
   said first and said second CRISPR endonucleases are configured to create single-strand DNA breaks,
a first single guide RNA (sgRNA), and
a second sgRNA,
   said first sgRNA is configured to hybridize to the sense strand of a genetic element controlling the expression of said gene, and
   said second sgRNA is configured to hybridize to the antisense strand of said genetic element controlling the expression of said gene.

According to the invention, a “targeted knockout” or “gene knockout” refers to a specific functional elimination of the expression of a gene on double stranded DNA by a genetic technique, however without destructing and/or affecting the coding region of said gene.

According to the invention “double-stranded DNA” (dsDNA) refers to a molecule of DNA consisting of two parallel polynucleotide chains joined by hydrogen bonds between complementary purines and pyrimidines.

According to the invention a “biological cell” includes both prokaryotic and eukaryotic cells, such as animal, plant or bacterial cells comprising DNA, in particular dsDNA. Human cells comprising dsDNA are of particular preference.

According to the invention, a “CRIPR endonuclease” refers to an enzyme which cleaves the phosphodiester bond within a polynucleotide chain and which is a component of a CRISPR (Clustered Regularly Interspaced Short Palindromic Repeat) complex. The CRISPR endonuclease is, therefore, capable to specifically bind to a guide or sgRNA which directs the endonuclease to a target nucleic acid. A CRISPR endonuclease is also referred to as ‘CRISPR associated protein’ (Cas) or CRISPR associated endonuclease, respectively.

According to the invention, in a first embodiment the first and second CRISPR endonucleases are identical, and in a second embodiment the first and second CRISPR endonucleases differ from each other.

According to the invention “a nucleic acid molecule encoding the CRISPR endonuclease” refers to a DNA, or cDNA or mRNA, which comprise or consist of the coding information for the synthesis of the CRISPR endonuclease.

According to the invention the first and second CRISPR endonucleases are configured to create single-strand DNA breaks on the target nucleic acid, but no double-strand DNA breaks, as typically carried out by naturally occurring CRISPR endonucleases, such as the types I, II, and IIIa. Such a configuration of the CRISPR endonucleases to be used in the invention can be achieved by a targeted modification of wild type CRISPR endonucleases. The modification typically addresses the catalytic center of the enzyme, thereby converting the nuclease into a nicking enzyme or “nickase”, respectively. As a consequence the modified CRISPR endonuclease according to the invention cuts one strand of a double-stranded DNA at a specific recognition nucleotide sequences known as a restriction site but is no longer capable to introduce DNA double-strand breaks. It hydrolyses (cuts) only one strand of the DNA duplex, to produce DNA molecules that are “nicked”, rather than cleaved.

According to the invention a “single guide RNA” (sgRNA) or guide RNA is a component of the CRISPR complex which serves to guide the CRISPR endonuclease to its target. The sgRNA is a non-coding short RNA sequence which binds to the complementary target DNA sequence. The sgRNA first binds to the CRISPR endonuclease enzyme and the sgRNA sequence guides the complex via pairing to a specific location on the DNA, where the modified CRISPR endonuclease performs its nickase activity by cutting the target single DNA strand.

According to the invention a “genetic element controlling the expression of said gene” refers to one or several regions located on the DNA bearing the target gene to be knocked-out, said region(s) control(s) the initiation of transcription of the gene. It is typically located near the transcription start of the gene, mostly upstream of the coding sequence. This region can be short (only a few nucleotides in length) or quite long (hundreds of nucleotides long). A typical genetic element controlling the expression of said gene according to the invention is a promoter.

According to the invention the first sgRNA comprises a nucleotide sequence which is essentially complementary to a sequence on the sense strand of the DNA bearing the coding sequence of said gene to be knocked-out. Said complementary sequence is located in a genetic element controlling the expression of said gene, typically upstream of the gene.

According to the invention the second sgRNA comprises a nucleotide sequence which is essentially complementary to a sequence on the antisense strand of the DNA bearing the coding sequence of said gene to be knocked-out. Said complementary sequence is located in a genetic element controlling the expression of said gene.

The essential complementary sequences of the first and second sgRNA allow the specific hybridization of the complex consisting of modified CRISPR endonuclease and sgRNA to the sense and antisense strand of the expression controlling element and the performance of a single-strand break thereof.

The inventors provide a genetic tool by means of which the targeted knockout of any gene on double-stranded DNA can be achieved in a reliable and safely manner. The safety profile of this approach is very high because the disruption of the controlling element, e.g. the promoter, occurs via the use of two CRISPR nickases with two sgRNAs. The nickase may be a mutated variant of a conventional CRISPR endonuclease, such as Cas9, that can only generated single-strand DNA breaks (SSB).

By using two guide RNAs or sgRNAs, respectively, as paired nickases, designed for the sense (plus) and antisense (minus) DNA strands, it is finally possible to create a double-strand break (DSB) by generating two single-strand breaks at the controlling element, e.g. the promoter upstream of the transcription start site of the gene to be knocked-out. The DSB would be created only if the two CRISPR endonucleases will cut simultaneously at the target sites. If any of the sgRNA will target any unwanted site in the genome and introduce a SSB non-specifically, there will be no adverse effects because the SSB will be repaired efficiently by the cellular repair machinery. Therefore, the composition according to the invention comprises one sgRNA that introduces a nick at the plus or sense strand of the controlling element upstream of the transcription start site (TSS) and one sgRNA that introduce the complementary nick at the minus or antisense strand upstream of the TSS. Simultaneous activity of both CRISPR endonulcease/sgRNA complexes lead to small genomic deletions in the controlling element, that permanently repress the gene transcription initiation complex.

The invention does not affect the coding sequence of the gene to be knocked-out in contrast to what is taught in the art, e.g. in Nasri et al. (2019, l.c.) or WO 2019/217294. The disruption of the coding sequence may result in malignant transformations. By using the invention the risk of malignant transformation is excluded or at least significantly reduced. Thus the composition of the invention is characterized by a very high safety profile which qualifies it for an administration in clinical routine applications.

The problem underlying the invention is herewith completely solved.

In an embodiment of the invention said genetic element controlling the expression of said gene is a promoter.

This measure has the advantage that such a controlling element is targeted which reliably and safely ensures the permanent knockout of the gene, however without affecting the coding region of the gene. According to the invention the promoter is a sequence of DNA to which proteins bind that initiate transcription of a single RNA from the DNA downstream of it. This transcribed RNA encodes the gene to be knocked-out. The promoter is typically located near the transcription start site of the gene, upstream on the DNA, i.e. towards the 5′ region of the sense strand. Promoters can be about 100-1000 base pairs long.

In another embodiment of the invention said first and/or second CRISPR endonuclease is a variant of the CRISPR associated protein 9 (Cas9).

This measure has the advantage that such CRISPR endonuclease is used for a conversion into a nickase, which is well established and characterized in the art. Cas9, formerly also called Cas5, Csn1, or Csx12, is a 160 kilodalton protein. It originates from the Gram-positive, aerotolerant bacterium Streptococcus pyogenes where it plays a vital role in the immunological defense against DNA viruses and plasmids. Streptococcus pyogenes utilizes CRISPR to memorize and Cas9 to later interrogate and cleave foreign DNA, such as invading bacteriophage DNA or plasmid DNA. Cas9 performs this interrogation by unwinding foreign DNA and checking for sites complementary to the 20 basepair spacer region of the guide RNA. If the DNA substrate is complementary to the guide RNA, Cas9 cleaves the invading DNA. Apart from its original function in bacterial immunity, the Cas9 protein has been heavily utilized as a genome engineering tool to induce site-directed double-strand breaks in DNA. Cas9 can cleave nearly any sequence complementary to the guide RNA. Because the target specificity of Cas9 stems from the guide RNA:DNA complementarity and not modifications to the protein itself, engineering Cas9 to target new DNA is straightforward. Native Cas9 requires a guide RNA composed of two disparate RNAs that associate—the CRISPR RNA (crRNA), and the trans-activating crRNA (tracrRNA). Cas9 targeting has been simplified through the engineering of a chimeric single guide RNA (chiRNA or sgRNA).

In another embodiment of the invention said variant of Cas9 comprises mutation(s) in the nuclease domain(s) RuvC and/or HNH, said mutation(s) conferring DNA nickase activity.

This measure has the advantage that the nickase conversion of Cas9 is effectively and reliably achieved by specifically addressing those catalytic domains in Cas9 which are responsible for the DNA cleavage activity. The mutations in the RuvC and/or HNH domains eliminate the activity of Cas9 to carry out DNA double strand breaks but establish the activity to effect DNA single-strand breaks.

In another embodiment of the invention said variant of Cas 9 is Cas9 D10A nickase and/or Cas9 H840A nickase.

This measure has the advantage that CRISPR endonucleases are employed, which are well established and characterized by their nicking enzyme activities which makes them predestined for a use in the invention. By the D10A amino acid exchange the RuvC catalytic center is disrupted, and by the H840A amino acid exchange the HNH catalytic domain is disrupted, both converting the enzyme into a nicking enzyme.

In another embodiment of the invention said gene is a mutated form of a wild type gene (mutated gene), preferably said mutated gene is subject of a gain-of-function mutation.

This measure has the advantage that the composition according to the invention is adapted to eradicate the phenotypical changes of a gene mutation, especially such a mutation which may play a causative gain-of-function role in hereditary diseases.

In view of the aforesaid, in another embodiment said mutated gene is a disease-associated gene.

An example of such a gain-of-function and disease-associated mutation is a mutation in the ELANE gene, such as the p.A57V CN mutation.

In a further embodiment said wild-type gene is a non-essential gene.

This measure has the advantage that the targeted knockout of the gene according to the invention can, on the one hand, avoid the consequences of the expression of a potential disease-associated gene, and, on the other hand, does not result in the loss of a vital function in the affected organism.

In another embodiment of the invention the mutated gene is a mutated form of the gene encoding neutrophil elastase or elastase neutrophil expressed gene (ELANE).

This measure has the advantage that a targeted knockout of such a gene is achieved which, if mutated, is considered of being responsible for most cases of severe congenital neutropenia (CN), and cyclic neutropenia (CyN). The inventors therefore provide a genetic tool for the effective treatment of disorders of the neutrophil production, which so far cannot be cured in a satisfying manner. An example is the p.A57V CN mutation in the ELANE gene.

In another embodiment of the composition of the invention said first and/or second sgRNA comprises a nucleotide sequence which is selected from the group consisting of SEQ ID NOS: 1 to 252.

This measure has the advantage that sgRNA sequences are used which demonstrably guide the CRISPR endonucleases to the ELANE promoter, allowing the latter to be simultaneously cut in the sense and the antisense DNA strands. Such simultaneous DNA single-strand breaks result in a DNA double strand break and a functional disruption of the promotor's function. This concerted action finally results in the inhibition of the expression of the ELANE gene.

Another subject-matter of the invention relates to the composition according to the invention for use as a medicament, preferably for use in the treatment of an autosomal-recessive disorder, alternatively an autosomal-dominantly inherited genetic disorder, further preferably for use in the treatment of congenital neutropenia, highly preferably afore-said disorders with gain-of-function mutations.

The features, characteristics, and embodiments of the composition according to the invention apply to the composition for use as a medicament and for use in said treatment according to the invention in a corresponding manner, even if not specifically indicated.

Another subject-matter of the invention relates to a method for the targeted knockout of a gene on double-stranded DNA in a biological cell, comprising the following steps:
1) providing a biological cell comprising a gene on double-stranded DNA;
2) introducing into said biological cell a composition comprising:
   a first CRISPR endonuclease or a nucleic acid molecule encoding the said CRISPR endonuclease;
   a second CRISPR endonuclease or a nucleic acid molecule encoding said second CRISPR endonuclease;
      said first and said second CRISPR endonucleases are configured to create single-strand DNA breaks,
   a first single guide RNA (sgRNA), and
   a second sgRNA,
      said first sgRNA is configured to hybridize to the sense strand of a genetic element controlling the expression of said gene, and
      said second sgRNA is configured to hybridize to the antisense strand of said genetic element controlling the expression of said gene, and
3) incubating said cell and said composition, thereby allowing the creation of a single-strand DNA break on the sense strand of a genetic element controlling the expression of said gene and a single-strand DNA break on the antisense strand of the genetic element controlling the expression of said gene.

The features, characteristics, and embodiments of the composition according to the invention apply to this method according to the invention in a corresponding manner.

Therefore, in an embodiment of this method said composition is the composition according to the invention.

In another embodiment of this method said biological cell is a primary cell, preferably a hematopoietic stem and progenitor cell (HSPC).

By this measure therapeutic cells are generated which can be used in an autologous stem cell transplantation or in vivo administration, respectively. The obtained HSPCs can be a component of a hematopoietic stem cell transplant (HSCT).

In another embodiment of the method according to the invention said biological cell originates from a subject with a mutated gene, further preferably said mutated gene is subject of a gain-of-function mutation, further preferably said mutated gene is a disease-associated gene, further preferably the wild-type counterpart gene of said mutated gene is a non-essential gene, and highly preferably said mutated gene is a mutated form of the gene encoding neutrophil elastase or the elastase neutrophil expressed gene (ELANE).

Another subject-matter relates to a preparation comprising a biological cell prepared in vitro by a method comprising the following steps:
1) providing a biological cell comprising a gene on double-stranded DNA;
2) introducing into said biological cell a composition comprising:
   a first CRISPR endonuclease or a nucleic acid molecule encoding the said CRISPR endonuclease;
   a second CRISPR endonuclease or a nucleic acid molecule encoding said second CRISPR endonuclease;
      said first and said second CRISPR endonucleases are configured to create single-strand DNA breaks,
   a first single guide RNA (sgRNA), and
   a second sgRNA,
      said first sgRNA is configured to hybridize to the sense strand of a genetic element controlling the expression of said gene, and
      said second sgRNA is configured to hybridize to the antisense strand of said genetic element controlling the expression of said gene;
3) incubating said cell and said composition, thereby allowing the creation of a single-strand DNA break on the sense strand of a genetic element controlling the expression of said gene and a single-strand DNA break on the antisense strand of the genetic element controlling the expression of said gene, and
4) recovering said cell.

The features, characteristics, and embodiments of the composition according to the invention and the method for the targeted knockout according to the invention apply to the preparation according to the invention in a corresponding manner. Again, the obtained cell, such as a hematopoietic stem and progenitor cell (HSPC), can be a component of a hematopoietic stem cell transplant (HSCT).

Therefore, in an embodiment said method used for the production of the preparation is the method according to the invention.

Another subject-matter of the invention is a kit for the targeted knockout of a gene on double-stranded DNA in a biological cell, comprising the composition according to the invention and instructions for delivering the composition to said biological cell so as to knockout the gene.

A “kit” is a combination of individual elements useful for carrying out the methods of the invention, wherein the elements are optimized for use together in the methods. The kits may also contain additional reagents, chemicals, buffers, reaction vials etc. which may be useful for carrying out the methods according to the invention. Such kits unify all essential elements required to work the methods according to the invention, thus minimizing the risk of errors. Therefore, such kits also allow semi-skilled laboratory staff to perform the methods according to the invention.

The features, characteristics, advantages and embodiments specified herein apply to the kit according to the invention, even if not specifically indicated.

Another subject-matter of the invention relates to a method of treating a subject afflicted with a disease associated with a mutated gene, comprising administration of a therapeutically effective amount of the composition according to the invention.

The features, characteristics, and embodiments of the composition according to the invention apply to this method according to the invention in a corresponding manner, even if not specifically indicated.

Another subject-matter of the invention relate to a nucleic acid molecule, preferably an RNA molecule, comprising a nucleotide sequence which is selected from the group consisting of SEQ ID NOS: 1 to 252.

Still another subject-matter of the invention relates to a method of treating a subject afflicted with a disease associated with a mutated gene, preferably a dominant-autosomal disease, comprising a step of inhibiting in said subject a genetic element controlling the expression of said gene, preferably knocking-out said genetic element controlling the expression of said gene.

The features, characteristics, and embodiments of the composition according to the invention apply to this method according to the invention in a corresponding manner, even if not specifically indicated.

It is to be understood that the before-mentioned features and those to be mentioned in the following cannot only be used in the combination indicated in the respective case, but also in other combinations or in an isolated manner without departing from the scope of the invention.

The invention is now further explained by means of embodiments resulting in additional features, characteristics and advantages of the invention. The embodiments are of pure illustrative nature and do not limit the scope or range of the invention. The features mentioned in the specific embodiments are general features of the invention which are not only applicable in the specific embodiment but also in an isolated manner and in the context of any embodiment of the invention.

The invention is now described and explained in further detail by referring to the following non-limiting examples and figures.

### BRIEF DESCRIPTION OF THE FIGURES

FIG. 1: Safe and efficient strategy to disrupt the ELANE gene expression by promoter knockout using CRISPR/Cas9 D10A nickase restores granulocytic differentiation of congenital neutropenia patient's HSPCs with p.A57V CN mutation. A, Scheme of the experiment. B, FACS gating strategy for cells generating during in vitro granulocytic differentiation C, Representative FACS density plots for granulocytic CD45⁺CD11b⁺CD15⁺, CD45⁺CD15⁺CD16⁺ and CD45⁺CD16b⁺C066b⁺ cell subsets of differentiated primary HSPCs of congenital neutropenia patient with p.A57V mutation after CRISPR/Cas9 D10A double nickases editing.

FIG. 2: ELANE promoter knockout restored granulocytic differentiation of congenital neutropenia patient's HSPCs with p.A57V ON mutation. A-D, Granulocytic differentiation capacity of ELANE promoter knockout HSPCs of congenital neutropenia patient's with p.A57V mutation was assessed by liquid culture differentiation after 13 days by investigating neutrophilic surface marker expression. Percentage (A), absolute cell counts (B), morphologic analysis of cytospin slides (C) and representative images of Wright-Giemsa stained cytopsins (D) are shown, E. Colony forming unit assay of the ELANE promoter knockout cells showed higher numbers of CFU-G compared to control cells. Data represent means±standard deviation (SD) from triplicates. *P<0.05, **P<0.01, ****P<0.0001.

FIG. 3: ELANE promoter knockout significantly impaired the ELANE gene expression and improved the functionality of differentiated neutrophils. A, ROS production was significantly elevated in promoter knockout cells treated with fMLP, as compared to non-target Cas9 cells. B, ELANE mRNA expression in in vitro generated CN neutrophils was 5-fold reduced compared to unedited healthy donor neutrophils. C, Indel's prevalence was assessed by inference of CRISPR edits (ICE) at day 13 post differentiation and was 87%.

FIG. 4: Independent in silico on- and off-target profiling of sgRNA confirms the safety of the selected sgRNAs. Independent in silica on- and off-target profiling of each sgRNA was done using Doench 2016 algorithm using CRISPRitz tool for up to 4 mismatches using reference genome GRCh37 (hg19) for sgRNA 2 (A) and sgRNA 30 (B) shows low off-target profile for each sgRNA.

FIG. 5: Analyzing ELANE promoter knockout genome editing outcome by Next generation sequencing. A, Quantification of editing frequency determined by the percentage and number of sequence reads showing unmodified and modified alleles. B, Nucleotide distribution across amplicon. At each base in the reference amplicon, the percentage of each base as observed in sequencing reads is shown (A=green; C=orange; G=yellow; T=purple). Black bars show the percentage of reads for which that base was deleted. Brown bars between bases show the percentage of reads having an insertion at that position. Sequence is listed as SEQ ID NO: 259. C, Combined frequency of any modification across the amplicon. Modifications outside of the quantification window are also shown.

FIG. 6: ELANE promoter knockout did NOT impair granulocytic differentiation of healthy control's HSPCs. Granulocytic differentiation capacity of ELANE promoter knockout HSPCs of healthy controls was assessed by liquid culture differentiation after 14 days by investigating neutrophilic surface marker expression and ELANE. PRTN3 and AZU1 mRNA expression in in vitro generated neutrophils from (A), healthy control 98 and (B), healthy control 99. Data represent means±standard deviation (SD) from triplicates. *P<0.05, **P<0.01, ****P<0.0001.

FIG. 7: Unbiased genome-wide profiling of off-target cleavage by CRISPR-Cas9 double nickase ELANE promoter knockout using GUIDE-seq. Profiling of off-target cleavage upon double nickase ELANE promoter knockout treatment using GUIDEseq showed only 2 potential off-target sites, LINC00992—Long Intergenic Non-Protein Coding RNA 992- and chr9:93703739—a non-coding intergenic region-. The ELANE promoter target sequence is shown in the top line with cleaved sites shown underneath and with mismatches to the on-target site shown and highlighted in color. GUIDE-seq sequencing read counts are shown to the right of each site. The on-target site is marked with a green circle and off-target sites with red circle in (A), sgRNA 30 (upper sequence “Reads” is listed as SEQ ID NO: 260) and (B), sgRNA 2 (upper sequence “Reads” is listed as SEQ ID NO: 261). All the genomic coordinates are based on reference genome GRCh37 (hg19).

### EXAMPLES

### 1. Material and Methods

### Patients

One severe congenital neutropenia patient harboring an ELANE mutation (ELANE-CN) was used in the study. Healthy individuals served as control group. Bone marrow and peripheral blood samples were collected from the patient in association with an annual follow-up recommended by the Severe Chronic Neutropenia International Registry. Study approval was obtained from the Ethical Review Board of the Medical Faculty, University of Tübingen. Informed written consent was obtained from the participant of this study.

### Cell Culture

Human CD34⁺ HSPC were isolated from bone marrow mononuclear cells by magnetic bead separation using human CD34 progenitor cell isolation kit. (Miltenyi Biotech, #130-046-703). CD34⁺ cells were cultured in a density of 2×10⁵ cells/ml in Stemline II hematopoietic stem cell expansion medium (Sigma Aldrich, #50192) supplemented with 10% FBS. 1% penicillin/streptomycin, 1% L-glutamine and a cytokine cocktail consisting of 20 ng/ml IL-3, 20 ng/ml IL-6. 20 ng/ml TPO, 50 ng/ml SCF and 50 ng/mL FLT-3L (all cytokines were purchased from R&D Systems).

### Design of the sgRNAs

Specific D10A nickase-guide-RNAs (sgRNAs) to target promoter region of ELANE gene (nick sites: chr19 [GGGCTATAAGAGGAGCCGGG (SEQ ID NO: 1; sgRNA 2), GAGGCCGTTGCATTGCCCCA (SEQ ID NO: 2; sgRNA 30)] were designed by using the Desktop genetics website. In total 252 sgRNAs specific for the ELANE promoter were developed by the inventors. They are listed in the following table along with their genomic positions according to the Genome Reference Consortium Human Build 37 (GRCh37), the specificity for the sense (1) or antisense strand (−1) and the protospacer adjacent motif (“PAM”).

TABLE 1
sgRNAs specific for the human
ELANE promoter

| | Genomic | | | |
|---|---|---|---|---|
| SEQ ID | position | | | |
| NO. | (hg19) | Strand | Sequence | PAM |
| 1 | 852285 | 1 | GGGCTATAAGAGGAGCCGGG | CGG |
| 2 | 852243 | −1 | GAGGCCGTTGCATTGCCCCA | CGG |
| 3 | 851023 | −1 | CTCACTTTCACCCAGGCTCA | CGG |
| 4 | 851030 | −1 | CGGGGAACTCACTTTCACCC | AGG |
| 5 | 851045 | 1 | GGTGAAAGTGAGTTCCCCGT | TGG |
| 6 | 851048 | 1 | GAAAGTGAGTTCCCCGTTGG | AGG |
| 7 | 851048 | −1 | CTCGTCTGTTGCCTCCAACG | GGG |
| 8 | 851049 | −1 | CCTCGTCTGTTGCCTCCAAC | GGG |
| 9 | 851050 | −1 | TCCTCGTCTGTTGCCTCCAA | CGG |
| 10 | 851060 | 1 | CCCGTTGGAGGCAACAGACG | AGG |
| 11 | 851065 | 1 | TGGAGGCAACAGACGAGGAG | AGG |
| 12 | 851069 | 1 | GGCAACAGACGAGGAGAGGA | TGG |
| 13 | 851073 | 1 | ACAGACGAGGAGAGGATGGA | AGG |
| 14 | 851078 | 1 | CGAGGAGAGGATGGAAGGCC | TGG |
| 15 | 851085 | −1 | AGGGCTCATTCTTGGGGGCC | AGG |
| 16 | 851090 | −1 | ACCTCAGGGCTCATTCTTGG | GGG |
| 17 | 851091 | −1 | AACCTCAGGGCTCATTCTTG | GGG |
| 18 | 851092 | −1 | GAACCTCAGGGCTCATTCTT | GGG |
| 19 | 851093 | −1 | TGAACCTCAGGGCTCATTCT | TGG |
| 20 | 851100 | 1 | GCCCCCAAGAATGAGCCCTG | AGG |
| 21 | 851104 | −1 | CAGCCGCTCCCTGAACCTCA | GGG |
| 22 | 851105 | −1 | CCAGCCGCTCCCTGAACCTC | AGG |
| 23 | 851106 | 1 | AAGAATGAGCCCTGAGGTTC | AGG |
| 24 | 851107 | 1 | AGAATGAGCCCTGAGGTTCA | GGG |
| 25 | 851112 | 1 | GAGCCCTGAGGTTCAGGGAG | CGG |
| 26 | 851116 | 1 | CCTGAGGTTCAGGGAGCGGC | TGG |
| 27 | 851126 | 1 | AGGGAGCGGCTGGAGTGAGC | CGG |
| 28 | 851134 | −1 | GCTGGACGGAGATCTGGGGC | CGG |
| 29 | 851138 | −1 | CGCAGCTGGACGGAGATCTG | GGG |
| 30 | 851139 | −1 | CCGCAGCTGGACGGAGATCT | GGG |
| 31 | 851140 | −1 | CCCGCAGCTGGACGGAGATC | TGG |
| 32 | 851148 | −1 | CTCTGGGACCCGCAGCTGGA | CGG |
| 33 | 851150 | 1 | CCCAGATCTCCGTCCAGCTG | CGG |
| 34 | 851151 | 1 | CCAGATCTCCGTCCAGCTGC | GGG |
| 35 | 851152 | −1 | AGGCCTCTGGGACCCGCAGC | TGG |
| 36 | 851160 | 1 | CGTCCAGCTGGGGGTOCCAG | AGG |
| 37 | 851164 | −1 | CGAGTGTAACCCAGGCCTCT | GGG |
| 38 | 851165 | 1 | AGCTGCGGGTCCCAGAGGCC | TGG |
| 39 | 851165 | −1 | GCGAGTGTAACCCAGGCCTC | TGG |
| 40 | 851166 | 1 | GCTGCGGGTCCCAGAGGCCT | GGG |
| 41 | 851172 | −1 | AGGAGCTGCGAGTGTAACCC | AGG |
| 42 | 851185 | 1 | TGGGTTACACTCGCAGCTCC | TGG |
| 43 | 851186 | 1 | GGGTTACACTCGCAGCTCCT | GGG |
| 44 | 851187 | 1 | GGTTACACTCGCAGCTCCTG | GGG |
| 45 | 851188 | 1 | GTTACACTCGCAGCTCCTGG | GGG |
| 46 | 851191 | 1 | ACACTCGCAGCTCCTGGGGG | AGG |
| 47 | 851192 | −1 | GCACGTCAAGGGCCTCCCCC | AGG |
| 48 | 851203 | −1 | TGGGAACTGAGGCACGTCAA | GGG |
| 49 | 851204 | −1 | TTGGGAACTGAGGCACGTCA | AGG |
| 50 | 851214 | −1 | GGGTTCCTGTTTGGGAACTG | AGG |
| 51 | 851220 | 1 | ACGTGCCTCAGTTOCCAAAC | AGG |
| 52 | 851222 | −1 | CCTTCCCAGGGTTCCTGTTT | GGG |
| 53 | 851223 | −1 | TCCTTCCCAGGGTTCCTGTT | TGG |
| 54 | 851228 | 1 | CAGTTCCCAAACAGGAACCC | TGG |
| 55 | 851229 | 1 | AGTTCCCAAACAGGAACCCT | GGG |
| 56 | 851233 | 1 | CCCAAACAGGAACCCTGGGA | AGG |
| 57 | 851234 | −1 | CACTTCTCTGGTCCTTOCCA | GGG |
| 58 | 851235 | −1 | GCACTTCTCTGGTCCTTCCC | AGG |
| 59 | 851246 | −1 | CTGCGCAATAGGCACTTCTO | TGG |
| 60 | 851257 | −1 | TCGGGCACTCACTGCGCAAT | AGG |
| 61 | 851275 | −1 | CGGCCACATGCAGCTGTGTC | GGG |
| 62 | 851276 | −1 | CCGGCCACATGCAGCTGTGT | CGG |
| 63 | 851283 | 1 | GTGCCCGACACAGCTGCATG | TGG |
| 64 | 851287 | 1 | CCGACACAGCTGCATGTGGC | CGG |
| 65 | 851295 | −1 | TACCCAGGGCCCTGTGATAC | CGG |
| 66 | 851296 | 1 | CTGCATGTGGCCGGTATCAC | AGG |
| 67 | 851297 | 1 | TGCATGTGGCCGGTATCACA | GGG |
| 68 | 851303 | 1 | TGGCCGGTATCACAGGGCCC | TGG |
| 69 | 851304 | 1 | GGCCGGTATCACAGGGCCCT | GGG |
| 70 | 851309 | −1 | CGCCTGCCTCAGTTTACCCA | GGG |
| 71 | 851310 | −1 | TCGCCTGCCTCAGTTTACCO | AGG |
| 72 | 851314 | 1 | ACAGGGCCCTGGGTAAACTG | AGG |
| 73 | 851318 | 1 | GGCCCTGGGTAAACTGAGGC | AGG |
| 74 | 851336 | 1 | GCAGGCGACACAGCTGCATG | TGG |
| 75 | 851340 | 1 | GCGACACAGCTGCATGTGGC | CGG |
| 76 | 851348 | −1 | TACCCAGGGCCCTGTGATAC | CGG |
| 77 | 851349 | 1 | CTGCATGTGGCCGGTATCAC | AGG |
| 78 | 851350 | 1 | TGCATGTGGCCGGTATCACA | GGG |
| 79 | 851356 | 1 | TGGCCGGTATCACAGGGCCC | TGG |
| 80 | 851357 | 1 | GGCCGGTATCACAGGGCCCT | GGG |
| 81 | 851362 | −1 | CGCCTGCCTCAGTTTACCCA | GGG |
| 82 | 851363 | −1 | TCGCCTGCCTCAGTTTACCC | AGG |
| 83 | 851367 | 1 | ACAGGGCCCTGGGTAAACTG | AGG |
| 84 | 851371 | 1 | GGCCCTGGGTAAACTGAGGC | AGG |
| 85 | 851389 | 1 | GCAGGCGACACAGCTGCATG | TGG |
| 86 | 851393 | 1 | GCGACACAGCTGCATGTGGC | CGG |
| 87 | 851401 | −1 | TACCCAGGGCCCTGTGATAC | CGG |
| 88 | 851402 | 1 | CTGCATGTGGCCGGTATCAC | AGG |
| 89 | 851403 | 1 | TGCATGTGGCCGGTATCACA | GGG |
| 90 | 851409 | 1 | TGGCCGGTATCACAGGGCCC | TGG |
| 91 | 851410 | 1 | GGCCGGTATCACAGGGCCCT | GGG |
| 92 | 851415 | −1 | CGCCTGCCTCAGTTTACCCA | GGG |
| 93 | 851416 | −1 | TCGCCTGCCTCAGTTTACCC | AGG |
| 94 | 851420 | 1 | ACAGGGCCCTGGGTAAACTG | AGG |
| 95 | 851424 | 1 | GGCCCTGGGTAAACTGAGGC | AGG |
| 96 | 851442 | 1 | GCAGGCGACACAGCTGCATG | TGG |
| 97 | 851454 | 1 | GCTGCATGTGGCCGTATCAC | AGG |
| 98 | 851454 | −1 | TTACCCAGGGCCCTGTGATA | CGG |
| 99 | 851455 | 1 | CTGCATGTGGCCGTATCACA | GGG |
| 100 | 851461 | 1 | GTGGCCGTATCACAGGGCCC | TGG |
| 101 | 851462 | 1 | TGGCCGTATCACAGGGCCCT | GGG |
| 102 | 851467 | −1 | CACCTGCCTCAGTTTACCCA | GGG |
| 103 | 851468 | −1 | TCACCTGCCTCAGTTTACCC | AGG |
| 104 | 851472 | 1 | ACAGGGCCCTGGGTAAACTG | AGG |
| 105 | 851476 | 1 | GGCCCTGGGTAAACTGAGGC | AGG |
| 106 | 851494 | 1 | GCAGGTGACACAGCTGCATG | TGG |
| 107 | 851498 | 1 | GTGACACAGCTGCATGTGGC | CGG |
| 108 | 851506 | 1 | GCTGCATGTGGCCGGTATCA | CGG |
| 109 | 851506 | −1 | TATCCAGGGCCCCGTGATAC | CGG |
| 110 | 851507 | 1 | CTGCATGTGGCCGGTATCAC | GGG |
| 111 | 851508 | 1 | TGCATGTGGCCGGTATCACG | GGG |
| 112 | 851514 | 1 | TGGCCGGTATCACGGGGCCC | TGG |
| 113 | 851520 | −1 | CGCCTGCCTCTGTTTATCCA | GGG |
| 114 | 851521 | −1 | TCGCCTGCCTCTGTTTATCC | AGG |
| 115 | 851525 | 1 | ACGGGGCCCTGGATAAACAG | AGG |
| 116 | 851529 | 1 | GGCCCTGGATAAACAGAGGC | AGG |
| 117 | 851547 | 1 | GCAGGCGACACAGCTGCATG | TGG |
| 118 | 851551 | 1 | GCGACACAGCTGCATGTGGC | CGG |
| 119 | 851559 | 1 | GCTGCATGTGGCCGGTATCA | CGG |
| 120 | 851559 | −1 | TACCCAGGGCCCCGTGATAC | CGG |
| 121 | 851560 | 1 | CTGCATGTGGCCGGTATCAC | GGG |
| 122 | 851561 | 1 | TGCATGTGGCCGGTATCACG | GGG |
| 123 | 851567 | 1 | TGGCCGGTATCACGGGGCCC | TGG |
| 124 | 851568 | 1 | GGCCGGTATCACGGGGCCCT | GGG |
| 125 | 851573 | −1 | CGCCTGCCTCAGTTTACCCA | GGG |
| 126 | 851574 | −1 | TCGCCTGCCTCAGTTTACCC | AGG |
| 127 | 851578 | 1 | ACGGGGCCCTGGGTAAACTG | AGG |
| 128 | 851582 | 1 | GGCCCTGGGTAAACTGAGGC | AGG |
| 129 | 851587 | 1 | TGGGTAAACTGAGGCAGGCG | AGG |
| 130 | 851599 | −1 | CCTGAGGGACTTGATGGGGG | TGG |
| 131 | 851602 | −1 | AGACCTGAGGGACTTGATGG | GGG |
| 132 | 851603 | −1 | TAGACCTGAGGGACTTGATG | GGG |
| 133 | 851604 | −1 | CTAGACCTGAGGGACTTGAT | GGG |
| 134 | 851605 | −1 | CCTAGACCTGAGGGACTTGA | TGG |
| 135 | 851610 | 1 | CCACCCCCATCAAGTCCCTC | AGG |
| 136 | 851614 | −1 | CCTGCCAAACCTAGACCTGA | GGG |
| 137 | 851615 | −1 | ACCTGCCAAACCTAGACCTG | AGG |
| 138 | 851616 | 1 | CCATCAAGTCCCTCAGGTCT | AGG |
| 139 | 851621 | 1 | AAGTCCCTCAGGTCTAGGTT | TGG |
| 140 | 851625 | 1 | CCCTCAGGTCTAGGTTTGGC | AGG |
| 141 | 851630 | 1 | AGGTCTAGGTTTGGCAGGTT | TGG |
| 142 | 851651 | 1 | GGCAAAAACACAGCAACGCT | CGG |
| 143 | 851668 | 1 | GCTCGGTTAAATCTGAATTT | CGG |
| 144 | 851669 | 1 | CTCGGTTAAATCTGAATTTC | GGG |
| 145 | 851683 | 1 | AATTTCGGGTAAGTATATCC | TGG |
| 146 | 851684 | 1 | ATTTCGGGTAAGTATATCCT | GGG |
| 147 | 851690 | −1 | GTCTCTTCCAAATGAGGCCC | AGG |
| 148 | 851694 | 1 | AGTATATCCTGGGCCTCATT | TGG |
| 149 | 851696 | −1 | ATCTAAGTCTCTTCCAAATG | AGG |
| 150 | 851735 | 1 | AAAAAACGTCGAGACCAGCC | CGG |
| 151 | 851738 | −1 | TTTCACCGTGTTGGCCGGGC | TGG |
| 152 | 851742 | −1 | GGGGTTTCACCGTGTTGGCC | GGG |
| 153 | 851743 | −1 | CGGGGTTTCACCGTGTTGGC | CGG |
| 154 | 851744 | 1 | CGAGACCAGCCCGGCCAACA | CGG |
| 155 | 851747 | −1 | GAGACGGGGTTTCACCGTGT | TGG |
| 156 | 851761 | −1 | TTGTATTTTTAGTAGAGACG | GGG |
| 157 | 851762 | −1 | TTTGTATTTTTAGTAGAGAC | GGG |
| 158 | 851763 | −1 | TTTTGTATTTTTAGTAGAGA | CGG |
| 155 | 851785 | 1 | AAAAATACAAAAAATTAGCC | AGG |
| 160 | 851792 | −1 | CAGGCGTGAGCCACTGCGCC | TGG |
| 161 | 851793 | 1 | AAAAAATTAGCCAGGOGCAG | TGG |
| 162 | 851811 | −1 | TCCCAGAGTGCTGGGATCAC | AGG |
| 163 | 851819 | −1 | CCTCAGCCTCCCAGAGTGCT | GGG |
| 164 | 851820 | 1 | CGCCTGTGATCCCAGCACTC | TGG |
| 165 | 851820 | −1 | GCCTCAGCCTOCCAGAGTGC | TGG |
| 166 | 851821 | 1 | GCCTGTGATCCCAGCACTCT | GGG |
| 167 | 851824 | 1 | TGTGATCCCAGCACTCTGGG | AGG |
| 168 | 851830 | 1 | CCCAGCACTCTGGGAGGCTG | AGG |
| 169 | 851834 | 1 | GCACTCTGGGAGGCTGAGGC | AGG |
| 170 | 851837 | 1 | CTCTGGGAGGCTGAGGCAGG | CGG |
| 171 | 851848 | 1 | TGAGGCAGGCGGATCACCCG | AGG |
| 172 | 851853 | −1 | GGTCTTGAACATCTGACCTC | GGG |
| 173 | 851854 | −1 | TGGTCTTGAACATCTGACCT | CGG |
| 174 | 851871 | 1 | TCAGATGTTCAAGACCAGCC | TGG |
| 175 | 851874 | −1 | TTTCGCCCTGTCGGCCAGGC | TGG |
| 176 | 851878 | −1 | AGTGTTTCGCCCTGTCGGCC | AGG |
| 177 | 851879 | 1 | TCAAGACCAGCCTGGCCGAC | AGG |
| 178 | 851880 | 1 | CAAGACCAGCCTGGCCGACA | GGG |
| 179 | 851883 | −1 | GAGACAGTGTTTCGCCCTGT | CGG |
| 180 | 851919 | 1 | CTACAAATACAAAAATTAGC | CGG |
| 181 | 851920 | 1 | TACAAATACAAAAATTAGCC | GGG |
| 182 | 851925 | 1 | ATACAAAAATTAGCCGGGAG | TGG |
| 183 | 851927 | −1 | CAGGCACCTGOCACCACTCC | CGG |
| 184 | 851928 | 1 | CAAAAATTAGCCGGGAGTGG | TGG |
| 185 | 851932 | 1 | AATTAGCCGGGAGTGGTGGC | AGG |
| 186 | 851946 | −1 | TCCTGAATAGCTGAGATTAC | AGG |
| 187 | 851956 | 1 | GCCTGTAATCTCAGCTATTC | AGG |
| 188 | 851959 | 1 | TGTAATCTCAGCTATTCAGG | AGG |
| 189 | 851965 | 1 | CTCAGCTATTCAGGAGGCTG | AGG |
| 190 | 851969 | 1 | GCTATTCAGGAGGCTGAGGC | AGG |
| 191 | 851987 | 1 | GCAGGAGAATCACTTGAACC | TGG |
| 192 | 851988 | 1 | CAGGAGAATCACTTGAACCT | GGG |
| 193 | 851991 | 1 | GAGAATCACTTGAACCTGGG | AGG |
| 194 | 851994 | 1 | AATCACTTGAACCTGGGAGG | CGG |
| 195 | 851994 | −1 | CACGGCAACCTCCGCCTCCC | AGG |
| 196 | 851997 | 1 | CACTTGAACCTGGGAGGCGG | AGG |
| 197 | 852011 | 1 | AGGCGGAGGTTGCCGTGAGC | CGG |
| 198 | 852012 | 1 | GGCGGAGGTTGCCGTGAGCC | GGG |
| 199 | 852012 | −1 | GGTGGCGTGATCCCGGCTCA | CGG |
| 200 | 852019 | −1 | GGAGTGCGGTGGCGTGATCC | CGG |
| 201 | 852030 | −1 | TCGCCCAGGCTGGAGTGCGG | TGG |
| 202 | 852033 | −1 | CTATCGCCCAGGCTGGAGTG | CGG |
| 203 | 852037 | 1 | CACGCCACCGCACTCCAGCC | TGG |
| 204 | 852038 | 1 | ACGCCACCGCACTCCAGCCT | GGG |
| 205 | 852040 | 1 | TCTTGCTCTATCGCCCAGGC | TGG |
| 206 | 852044 | −1 | AGAGTCTTGCTCTATCGCCC | AGG |
| 207 | 852071 | −1 | GGTTTTTTÅATTTATTTTTT | TGG |
| 208 | 852092 | −1 | AAATGTCAGATAATCAATGT | GGG |
| 209 | 852093 | −1 | CAAATGTCAGATAATCAATG | TGG |
| 210 | 852131 | −1 | GGGGCCTCCAGACAAAATTC | AGG |
| 211 | 852135 | 1 | TGTGCATCCTGAATTTTGTC | TGG |
| 212 | 852138 | 1 | GCATCCTGAATTTTGTCTGG | AGG |
| 213 | 852150 | −1 | ACGCTGGATTGGCTCGGGTG | GGG |
| 214 | 852151 | −1 | GACGCTGGATTGGCTCGGGT | GGG |
| 215 | 852152 | −1 | AGACGCTGGATTGGCTCGGG | TGG |
| 216 | 852155 | −1 | ACAAGACGCTGGATTGGCTC | GGG |
| 217 | 852156 | −1 | GACAAGACGCTGGATTGGCT | CGG |
| 218 | 852161 | −1 | AGGGGGACAAGACGCTGGAT | TGG |
| 219 | 852166 | −1 | GGAGAAGGGGGACAAGACGC | TGG |
| 220 | 852178 | −1 | TGATGAAAAGGGGGAGAAGG | GGG |
| 221 | 852179 | −1 | TTGATGAAAAGGGGGAGAAG | GGG |
| 222 | 852180 | −1 | GTTGATGAAAAGGGGGAGAA | GGG |
| 223 | 852181 | −1 | CGTTGATGAAAAGGGGGAGA | AGG |
| 224 | 852187 | −1 | ACAGGGCGTTGATGAAAAGG | GGG |
| 225 | 852188 | −1 | CACAGGGCGTTGATGAAAAG | GGG |
| 226 | 852189 | −1 | GCACAGGGCGTTGATGAAAA | GGG |
| 227 | 852190 | −1 | GGCACAGGGCGTTGATGAAA | AGG |
| 228 | 852204 | 1 | TTTCATCAACGCCCTGTGCC | AGG |
| 229 | 852204 | −1 | ACTTCCTCTCCCCTGGCACA | GGG |
| 230 | 852205 | 1 | TTCATCAACGCCCTGTGCCA | GGG |
| 231 | 852205 | −1 | CACTTCCTCTCCCCTGGCAC | AGG |
| 232 | 852206 | 1 | TCATCAACGCCCTGTGCCAG | GGG |
| 233 | 852211 | 1 | AACGCCCTGTGCCAGGGGAG | AGG |
| 234 | 852211 | −1 | GCCCTCCACTTCCTCTCCCC | TGG |
| 235 | 852217 | 1 | CTGTGCCAGGGGAGAGGAAG | TGG |
| 236 | 852220 | 1 | TGCCAGGGGAGAGGAAGTGG | AGG |
| 237 | 852221 | 1 | GCCAGGGGAGAGGAAGTGGA | GGG |
| 238 | 852227 | 1 | GGAGAGGAAGTGGAGGGCGC | TGG |
| 239 | 852231 | 1 | AGGAAGTGGAGGGCGCTGGC | CGG |
| 240 | 852237 | 1 | TGGAGGGCGCTGGCCGGCCG | TGG |
| 241 | 852238 | 1 | GGAGGGCGCTGGCCGGCCGT | GGG |
| 242 | 852239 | 1 | GAGGGCGCTGGCCGGCCGTG | GGG |
| 243 | 852239 | −1 | CCGTTGCATTGCCCCACGGC | CGG |
| 244 | 852250 | 1 | CCGGCCGTGGGGCAATGCAA | CGG |
| 245 | 852262 | −1 | TCTTATAGCCCTGTGCTGGG | AGG |
| 246 | 852264 | 1 | ATGCAACGGCCTCCCAGCAC | AGG |
| 247 | 852265 | 1 | TGCAACGGCCTCCCAGCACA | GGG |
| 248 | 852265 | −1 | TCCTCTTATAGCCCTGTGCT | GGG |
| 249 | 852266 | −1 | CTCCTCTTATAGCCCTGTGC | TGG |
| 250 | 852275 | 1 | TCCCAGCACAGGGCTATAAG | AGG |
| 251 | 852281 | 1 | CACAGGGCTATAAGAGGAGC | CGG |
| 252 | 852282 | 1 | ACAGGGCTATAAGAGGAGCC | GGG |

### CRISPR/Cas9 D10A Nickase-gRNA RNP Mediated ELANE Promoter KO in HSPC

Nucleofection was carried out using the Amaxa nucleofection system (P3 primary kit, #V4XP-3024) according to the manufacturer's instructions. For 1×10⁶ human CD34⁺ HSPC, 300 pmol sgRNA and 300 pmol Hifi Cas9 protein (Integrated DNA Technologies) was used for nucleofection.

### Assessing Genome Editing Efficiency

Gene editing efficiency assessed either by Sanger sequencing of the promoter region qRT-PCR.

### Assessing Gene Editing Efficiency by NGS

rhAmpSeq CRISPR analysis system approach was used to accurately estimate the on-target editing efficiency. The IDT's rhAmpSeq design tool was used to design rhAMP PCR primers to amplify the target area in ELANE promoter. The PCR amplification followed by rhAmpSeq library preparation was performed using rhAmpSeq CRISPR Library Kit (IDT, #10007317) according to manufacturer's protocol. The rhAmpSeq libraries was sequenced on an Illumina platform by Novogene. The NGS data was analysed by IDT's rhAmpSeq CRISPR analysis pipeline followed by CRISPRESSO tool.

### RNA Isolation, cDNA Synthesis and qRT-PCR

RNA was isolated using RNeasy Mini Kit (Qiagen) and cDNA was prepared using Omniscript RT kit (Qiagen). qPCR was performed using SYBR Green qPCR master mix (Roche) and Light Cycler 480 (Roche). ELANE [primers: Fwd: GTGTCWTCCTCGCCTGTGTC (SEQ ID NO: 253), Rev: CCCACAATCTCCGAGGCCAG (SEQ ID NO: 254)] mRNA expression was normalized to GAPDH [primers: Fwd: CTGGGCTACACTGAGCACC (SEQ ID NO: 255), Rev: AAGTGGTCGTTGAGGGCAATG (SEQ ID NO: 256)] and for ACTB [primers: Fwd: CATGTACGTTGCTATCCAGGC (SEQ ID NO: 257), Rev: CTCCTTAATGTCACGCACGAT (SEQ ID NO: 258)] mRNA expression levels.

### GUIDEseq Off-Target Profiling

GUIDEseq was used to detect potential off-target cleavage sites after CRISPR/Cas9 editing. Primary human bone-marrow derived CD34+ cells were cultured in a density of 2×105 cells/mL in Stemline II hematopoietic stem cell expansion medium (Sigma Aldrich, #50192) supplemented with 10% FBS, 1% penicillin/streptomycin, 1% L-glutamine and a cytokine cocktail consisting of 20 ng/mL IL-3, 20 ng/mL IL-6, 20 ng/mL TPO, 50 ng/ml SCF and 50 ng/mL FLT-3L (all cytokines were purchased from R&D Systems). CD34+ cells were electroporated with 8 μg Cas9 D10A Nickase V3 (IDT, #1081063) assembled with 195 ng sgRNA 2 (IDT) and 195 ng sgRNA 30 (IDT). dsODN was added at a final concentration of 1.5 μM (5″-P-G*T*TTAATTGAGTTGTCATATGTTAATAACGGT*A*T-3′ (SEQ ID 262), 5-P-A*T*ACCGTTATTAACATATGACAACTCAATTAA*A*C -3′ (SEQ ID 263) using the electroporation program CA-137 in 20 μl P3 buffer (Lonza, #V4XP-3032) on a Lonza 4-D nucleofector device according to the manufacturer's instruction. 96 hours post-electroporation, the genomic DNA was isolated using QIAamp DNA Blood Mini Kit (Qiagen, #51104) according to manufacturers recommendations. Isolated DNA was sheared with Covaris S200 instrument to an average length of DNA pieces of 500 bp. Sheared DNA was end-repaired, A-tailed and ligated to half-functional adapters, incorporating a 8-nt random molecular index. Two rounds of nested anchored PCR with primers complementary to the oligo tag were used for target enrichment. The final library was sequenced on an Illumina platform by Novogene. The NGS results were analyzed by GUIDEseq analysis pipeline.

### Liquid Culture differentiation of CD34+ Cells

CD34+ cells were seeded in a density of 2×10⁵ cells/ml. Cells were incubated for 7 days in RPMI 1640 GlutaMAX supplemented with 10% FBS. 1% penicillin/streptomycin, 5 ng/ml SCF, 5 ng/ml IL-3, 5 ng/ml GM-CSF and 10 ng/ml G-CSF. Medium was exchanged every second day. On day 7, cells were plated in RPMI 1640 GlutaMAX supplemented with 10% FBS, 1% penicillin/streptomycin and 10 ng/ml G-CSF. On day 14, cells were analyzed by FACS using following mouse anti-human anti-bodies: CD34 (BB, #343811). CD33 (BioLegend, #303416), CD45 (BioLegend, #304036), CD11 b (BD, #557754), CD15 (BE, #555402), CD16 (BD, #561248), CD66b (Biolegend, #305104). Morphology of the cells was investigated on cytospin slides by Wright-Giemsa staining.

### 2. Results

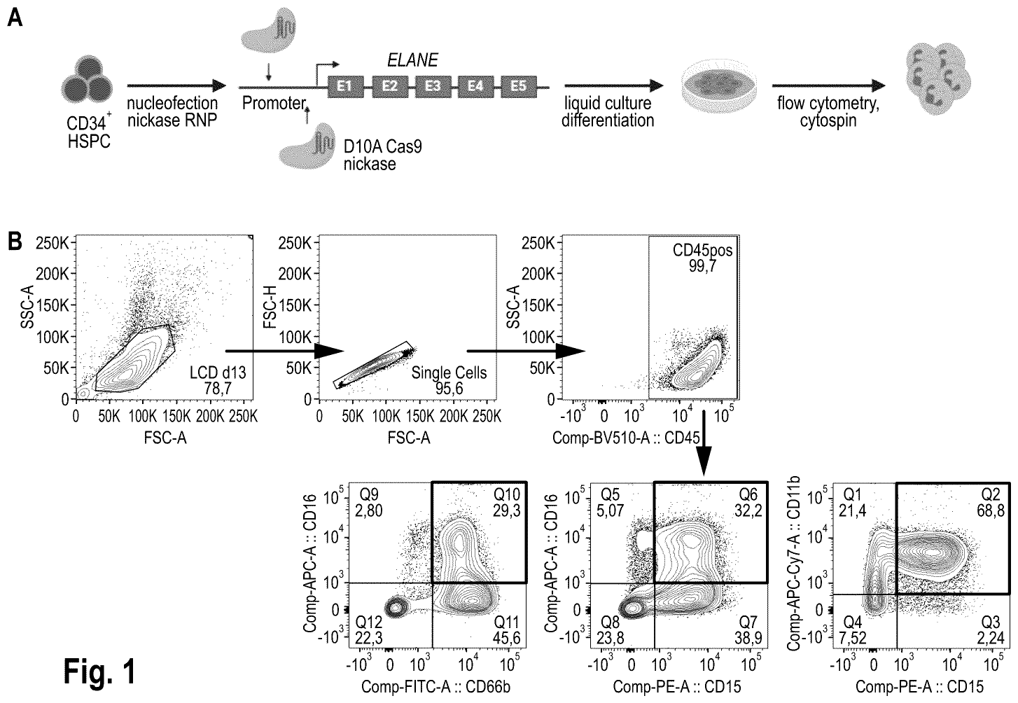

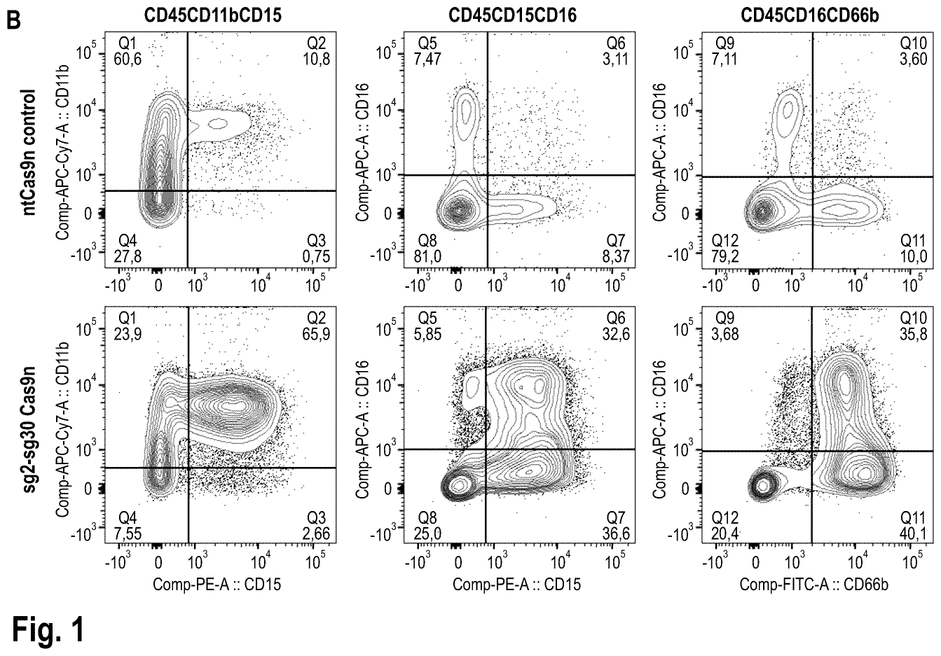

To perform ELANE transcriptional repression using CRISPR/Cas9 D10A nickase, HSPCs of an ELANE-CN patient with a p.A57V mutation were nucleofected with CRISPR/Cas9 D10A RNP and two days later cells were processed for liquid culture differentiation towards neutrophils (FIG. 1A). The same approach was used with healthy volunteers (data not shown).

Using the double nick strategy in ELANE-CN HSPCs, the inventors observed markedly elevated neutrophil differentiation, as assessed by the percentage of CD45⁺CD11b⁺CD15⁺, CD45⁺CD15⁺CD16⁺ and CD45⁺CD16⁺CD66b⁺ cells (FIGS. 1B, C and 2A, B) compared to ELANE-CN control cells nucleofected with non-targeting CRISPR/Cas9 D10A RNP. No effects were observed with healthy volunteers (data not shown).

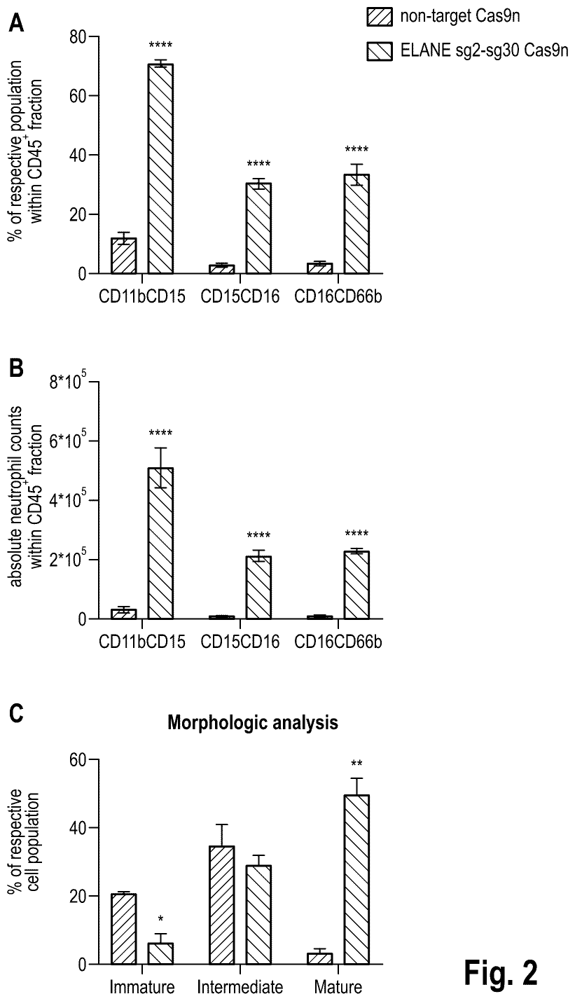

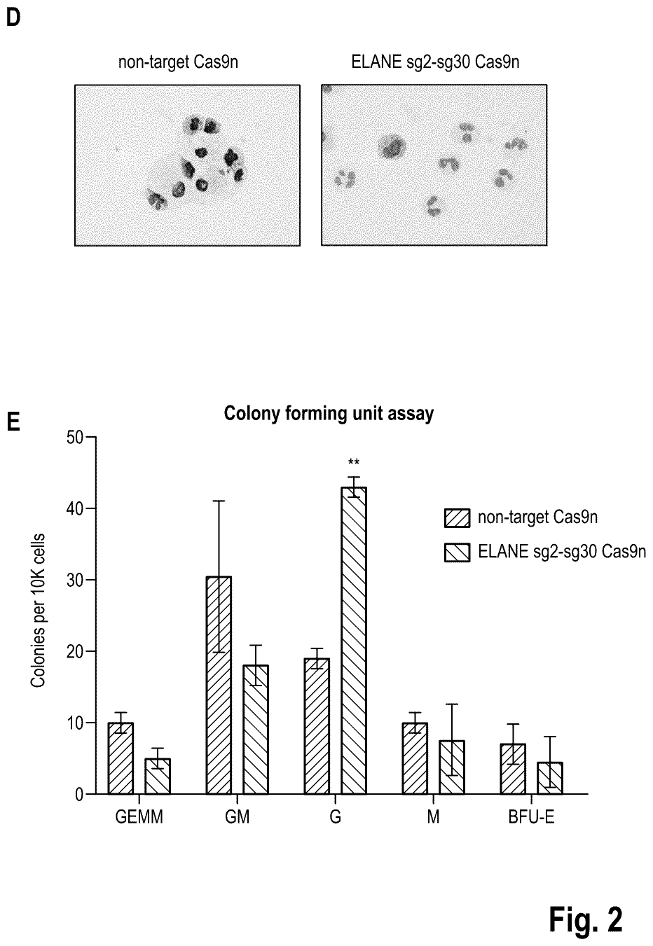

Again, comparing morphological analysis of ELANE-CN with promoter KO to control cells (see above) the strongly improved number of mature granulocytes was confirmed (FIG. 2C, D).

Similar results were achieved by colony forming unit assay (CFU) showing significantly more granulocytic colony-forming units (CFU-G colonies) in the ELANE promoter KO cells compared to non-targeting CRISPR/Cas9 D10A RNP control (FIG. 2E). Again, effects were observed with healthy volunteers (data not shown).

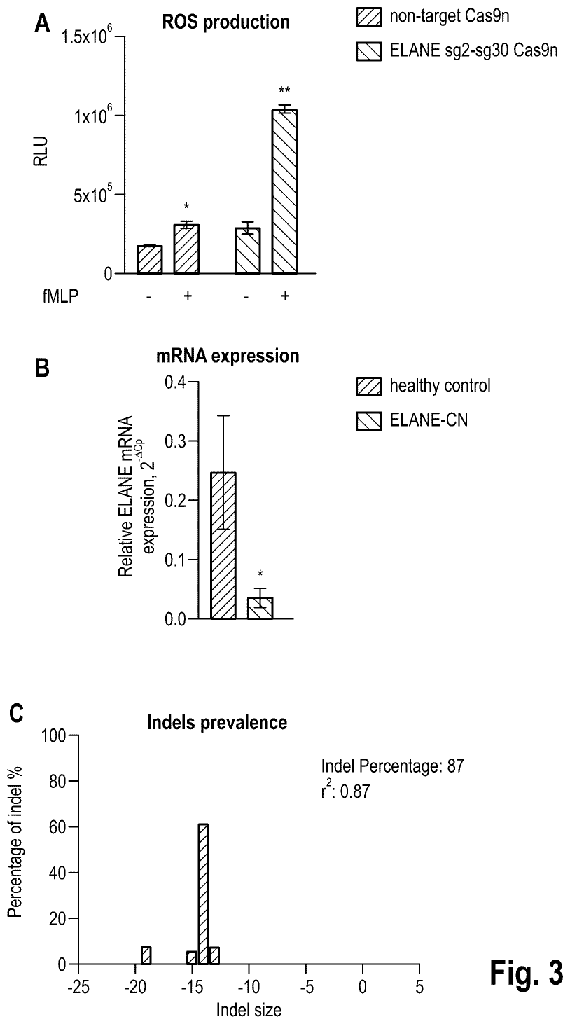

The inventors further tested the functionality of in vitro generated ELANE promoter KO neutrophils by testing the reactive oxygen species (ROS) production in response to fMLP. Indeed, ELANE promoter KO cells produced significantly more ROS) in response to fMLP, as compared to control cells with non-targeting CRISPR/Cas9 D10A RNP (FIG. 3A).

ELANE mRNA expression was reduced by around 5-fold in neutrophils from ELANE promoter KO cells compared to neutrophils from healthy donors (FIG. 3B). Indel's prevalence at targeted site was 87% as assessed by inference of CRISPR edits (ICE) from Sanger Sequencing Trace Data (FIG. 3C), indicating high gene-editing efficiency.

No effects were observed with healthy volunteers (data not shown).

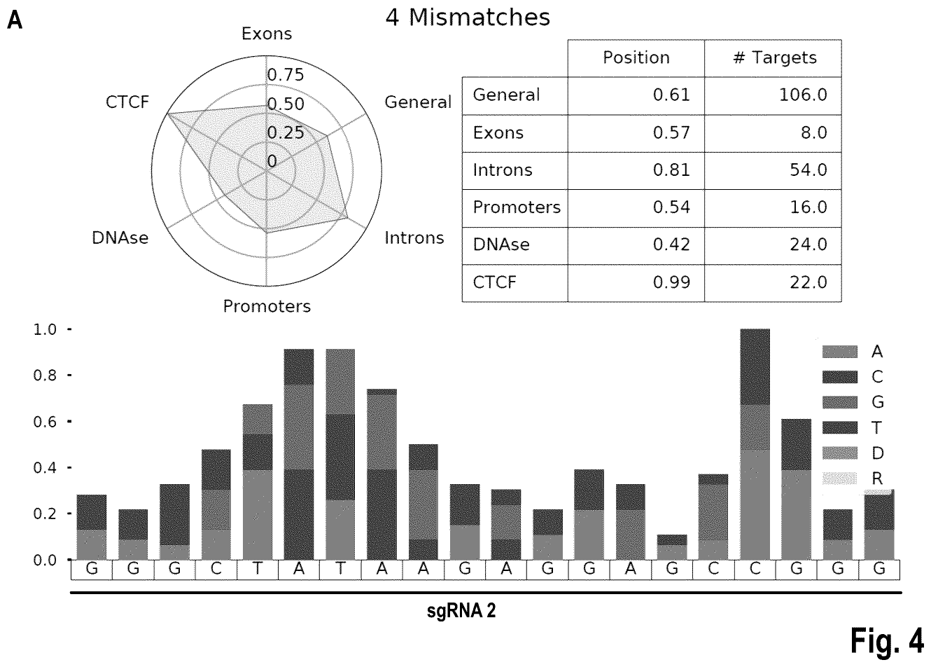

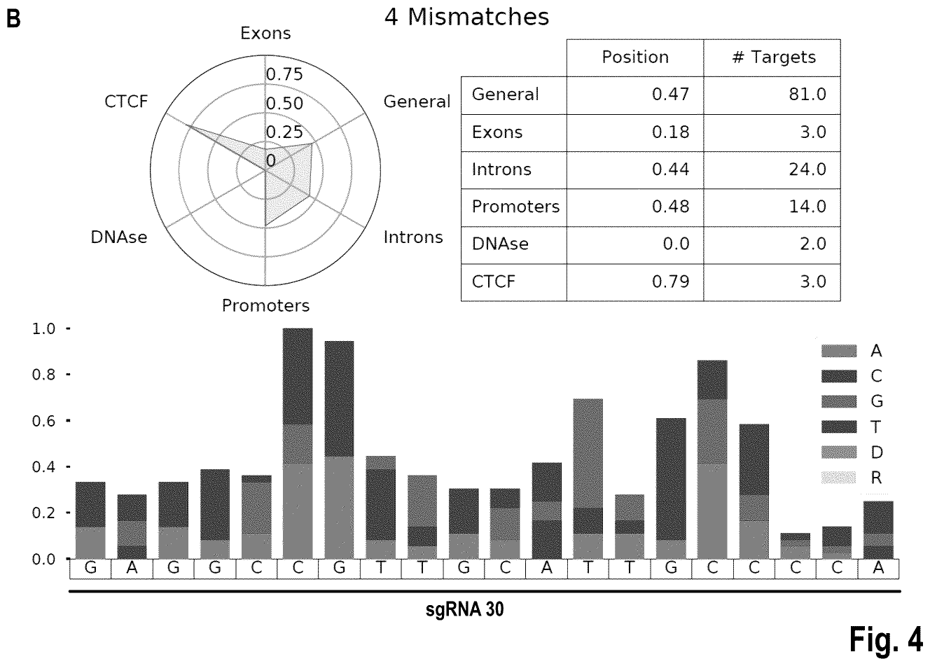

The inventors also performed an independent in silica on- and off-target profiling of each sgRNA for up to 4 mismatches using Doench 2016 algorithm of CRISPRitz tool (FIG. 4A, B). The inventors found low off-target effects for each single sgRNAs, indicating very low off-target effects of the combination of both sgRNAs (FIG. 4A, B).

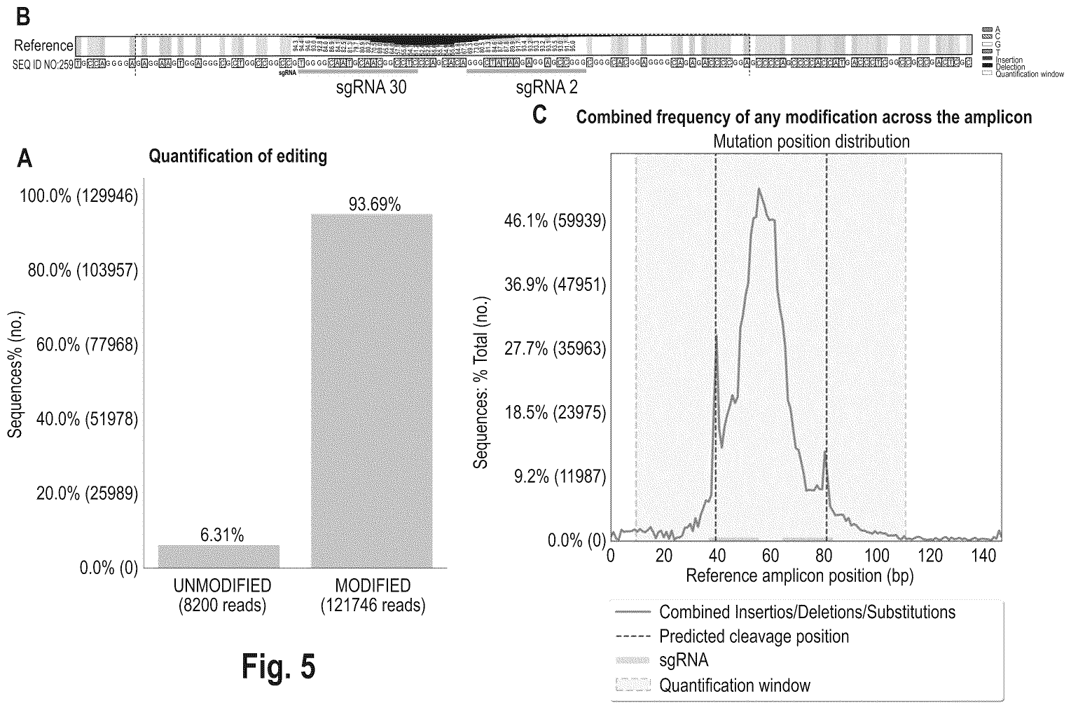

To further obtain an NGS grade resolution on on-target profile of editing outcome, the inventors used rhAmpSeq CRISPR Analysis System. They found that the editing efficiency was 93%. In FIG. 5A, B, the nucleotide composition across the edited region is depicted along with the frequency of any modification across the edited site.

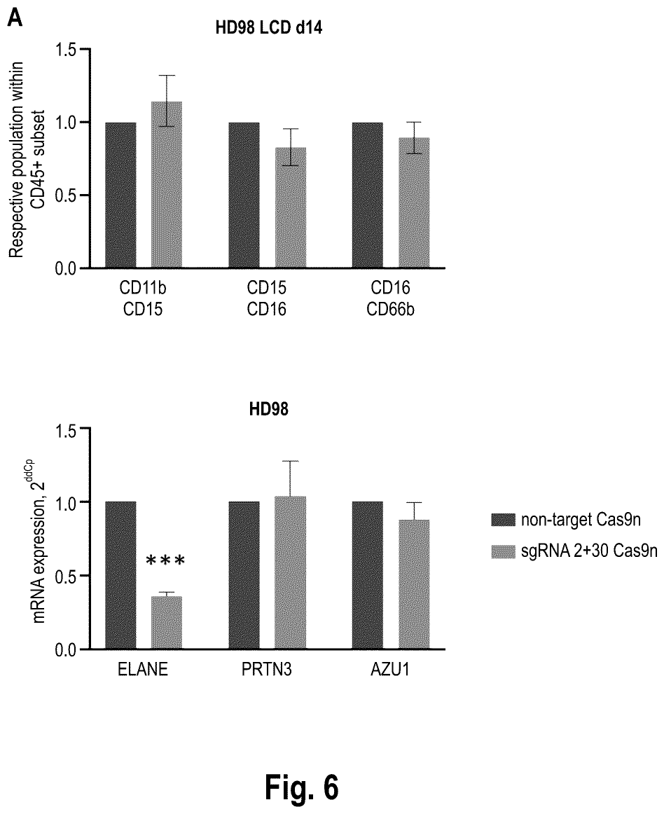

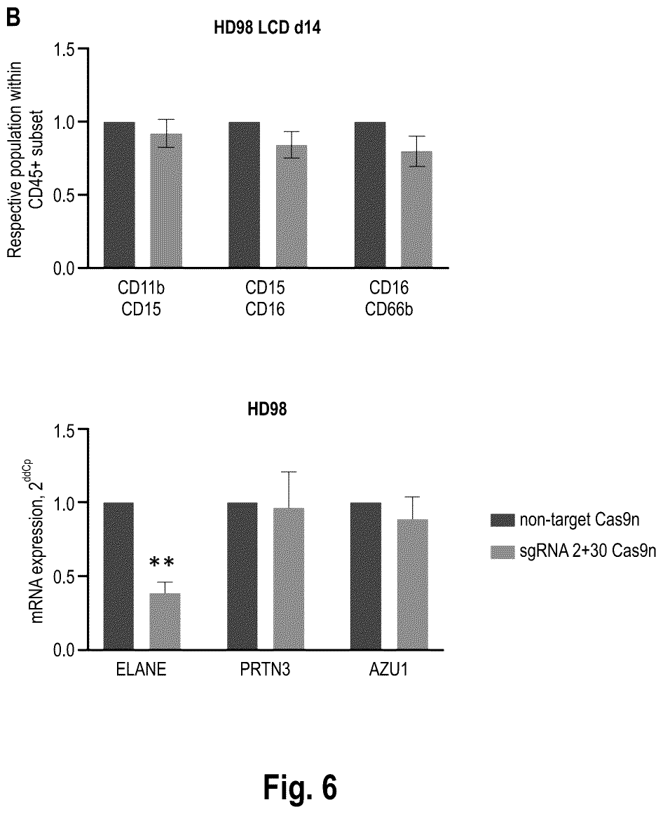

To investigate if the inventors' proposed CRISPR/Cas9 strategy may affect granulocytic differentiation, HSPCs of two healthy controls were nucleofected with CRISPR/Cas9 D10A RNP and two days later cells were processed for liquid culture differentiation towards neutrophils. The inventors observed no impairment in neutrophil differentiation of edited cells, as assessed by the percentage of CD45+CD11b+CD15+, CD45+CD15+CD16+ and CD45+CD16+CD66b+ neutrophilic cells that was comparable to healthy control cells nucleofected with non-targeting CRISPR/Cas9 D10A RNP (FIG. 6A, B). While ELANE mRNA expression was reduced significantly in neutrophils from ELANE promoter KO cells, compared to neutrophils from healthy donors nucleofected with non-targeting CRISPR/Cas9 D10A RNP, the inventors observed no changes in mRNA expression levels of two NE related serine proteases, PRTN3 and AZU1 (FIG. 6A, B).

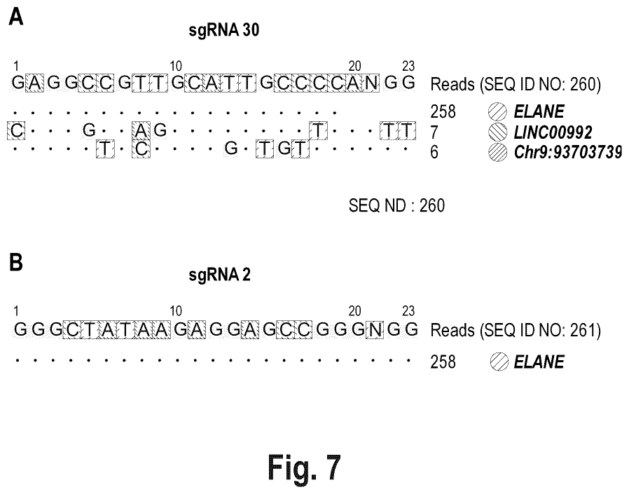

Therapeutic use of CRISPR/Cas9 in clinic needs a comprehensive knowledge of potential off-target effects to minimize the risk of harmful consequences. Therefore, the inventors performed GUIDE-seq analysis, to be able to have an unbiased genome-wide profiling of off-target cleavage by CRISPR/Cas9 D10A double nickase targeting the ELANE gene promoter. The inventors observed two potential off-target sites, LINC00992-Long Intergenic Non-Protein Coding RNA 992- and Chr9:93703739—a noncoding intergenic region—which may happen if the RNA guided nuclease had 6 or 7 mismatches with the genome. The GUIDE-seq results highlights the safety profile of ELANE promoter editing approach for clinic (FIG. 7A, B).

### 3. Conclusion

Taken together, the inventors established a safe approach for reducing a gene's expression by CRISPR/Cas9 double nick strategy without affecting the gene's coding sequence. This approach was exemplified as feasible using the ELANE gene involved in the development of congenital neutropenia. As demonstrated by examples the approach according to the invention can rescue granulopoiesis in ELANE-CN patients. In healthy individuals the knock-out of the ELANE promoter has no effect on granulopoiesis. Therefore, the invention offers a long-term treatment option replacing the daily G-CSF therapy and decreasing the risk of leukemia development.

Neutrophil elastase (NE) is a proteolytic enzyme of the neutrophil serine protease (NSP) family, including also cathepsin G (CG), proteinase 3 (PR3) and azurocidin (AZU1). NSPs are stored in cytoplasmic granules, can be secreted into the extra- and peri-cellular space upon cellular activation and considered to be crucially involved in bacterial defense. Elane^{-/-} mice have normal neutrophil counts, but there are conflicting results regarding the effect of NE-deficiency on neutrophil extravasation to sites of inflammation, phagocytosis, and neutrophil extracellular traps in mice. NE may or may not be essential for these processes. Papillon-Lefevre Syndrome (PLS) is a human disorder known to cause NE deficiency. This rare autosomal recessive disease is due to loss-of-function mutations in the DPPI gene locus with the loss of the lysosomal cysteine protease cathepsin C/dipeptidyl peptidase I (DPPI). The activation NSP, including NE, depends on the N-terminal processing activity of DPPI. Therefore, PLS patients exhibit a severe reduction in the activity and stability of all three NSP. Intriguingly, patients with PLS have no defects in their ability to kill bacteria e.g. Staphylococcus aureus or Escherichia coli, suggesting that redundancies in the neutrophil's bactericidal mechanisms negate the necessity for serine proteases for killing common bacteria. Based on these observations, at this juncture, the inventors believe that CRISPR/Cas9 based knockout of ELANE in HSPC of CN patients may restore defective granulopoiesis in CN patients without seriously impairing neutrophil functions.

What is claimed is:

1. A composition for the targeted knockout of a gene on double-stranded DNA in a biological cell, comprising:
a first CRISPR endonuclease or a nucleic acid molecule encoding the said CRISPR endonuclease;
a second CRISPR endonuclease or a nucleic acid molecule encoding said second CRISPR endonuclease;
said first and said second CRISPR endonucleases are configured to create single-strand DNA breaks,
a first single guide RNA (sgRNA), and
a second sgRNA,
said first sgRNA is configured to hybridize to the sense strand of a genetic element controlling the expression of said gene, and
said second sgRNA is configured to hybridize to the antisense strand of said genetic element controlling the expression of said gene.

2. The composition of claim 2, wherein said genetic element controlling the expression of said gene is a promoter.

3. The composition of claim 1, wherein said first or second CRISPR endonuclease is a variant of CRISPR associated protein 9 (Cas9).

4. The composition of claim 3, wherein said variant of Cas9 comprises mutation(s) in the nuclease domain(s) RuvC or HNH, said mutation(s) conferring DNA nickase activity.

5. The composition of claim 3, wherein said variant of Cas 9 is Cas9 D10A nickase or Cas9 H840A nickase.

6. The composition of claim 1, wherein said gene is a mutated form of a wild type gene (mutated gene).

7. The composition of claim 6, wherein said mutated gene is selected from the group consisting of: gain-of-function mutated gene, disease-associated mutated gene, mutated non-essential gene, and a mutated form of the gene encoding neutrophil elastase (ELANE).

8. The composition of claim 1, wherein said first or second sgRNA comprises the nucleotide sequence which is selected from the group consisting of SEC ID NOS: 1 to 252.

9. A method for the targeted knockout of a gene on double-stranded DNA in a biological cell, comprising the following steps:
1) providing a biological cell comprising a gene on double-stranded DNA;
2) introducing into said biological cell a composition comprising:
a first CRISPR endonuclease or a nucleic acid molecule encoding the said CRISPR endonuclease;
a second CRISPR endonuclease or a nucleic acid molecule encoding said second CRISPR endonuclease;
said first and said second CRISPR endonucleases are configured to create single-strand DNA breaks,
a first single guide RNA (sgRNA), and
a second sgRNA,
said first sgRNA is configured to hybridize to the sense strand of a genetic element controlling the expression of said gene, and
said second sgRNA is configured to hybridize to the antisense strand of said genetic element controlling the expression of said gene, and
3) incubating said cell and said composition, thereby allowing the creation of a single-strand DNA break on the sense strand of a genetic element controlling the expression of said gene and a single-strand DNA break on the antisense strand of the genetic element controlling the expression of said gene.

10. The method of claim 9, wherein said composition is the composition of claim 1.

11. The method of claim 9, wherein said biological cell is a primary cell or a hematopoietic stem and progenitor cell (HSPC).

12. The method of claim 11, wherein said biological cell originates from a subject with a mutated gene.

13. The method of claim 12, wherein said mutated gene is selected from the group consisting of: gain-of-function mutated gene, disease-associated mutated gene, mutated non-essential gene, and mutated form of the gene encoding neutrophil elastase (ELANE).

14. A preparation comprising a biological cell prepared in vitro by a method comprising the following steps:
1) providing a biological cell comprising a gene on double-stranded DNA;
2) introducing into said biological cell a composition comprising:
a first CRISPR endonuclease or a nucleic acid molecule encoding the said CRISPR endonuclease;
a second CRISPR endonuclease or a nucleic acid molecule encoding said second CRISPR endonuclease;
said first and said second CRISPR endonucleases are configured to create single-strand DNA breaks,
a first single guide RNA (sgRNA), and
a second sgRNA,
said first sgRNA is configured to hybridize to the sense strand of a genetic element controlling the expression of said gene, and
said second sgRNA is configured to hybridize to the antisense strand of said genetic element controlling the expression of said gene;
3) incubating said cell and said composition, thereby allowing the creation of a single-strand DNA break on the sense strand of a genetic element controlling the expression of said gene and a single-strand DNA break on the antisense strand of the genetic element controlling the expression of said gene, and
4) recovering said cell.

15. A kit for the targeted knockout of a gene on double-stranded DNA in a biological cell, comprising the composition of claim 1 and instructions for delivering the composition to said biological cell so as to knockout the gene.

16. A method of treating a subject afflicted with a disease associated with a mutated gene, comprising administration of a therapeutically effective amount of the composition of claim 1.

17. A nucleic acid molecule comprising a nucleotide sequence which is selected from the group consisting of SEQ ID NOS: 1 to 252.

18. The nucleic acid molecule of claim 17, which is an RNA molecule.

19. A method of treating a subject afflicted with a disease associated with a mutated gene, comprising inhibiting in said subject a genetic element controlling the expression of said gene.

20. The method of claim 19, wherein said inhibiting is carried out by knocking-out said genetic element controlling the expression of said gene.

21. The method of claim 19, wherein said subject is afflicted with a dominant-autosomal disease or an autosomal-recessive disorder.
